(19) United States
(12) Patent Application Publication
(10) Pub. No.: US 2018/0088079 A1
(43) Pub. Date: Mar. 29, 2018

(54) NANOSCALE WIRES WITH EXTERNAL LAYERS FOR SENSORS AND OTHER APPLICATIONS

(71) Applicant: President and Fellows of Harvard College, Cambridge, MA (US)

(72) Inventors: Charles M. Lieber, Lexington, MA (US); Ning Gao, Cambridge, MA (US); Wei Zhou, Blacksburg, VA (US); Xiaocheng Jiang, Cambridge, MA (US); Teng Gao, Somerville, MA (US); Xiao Yang, Cambridge, MA (US)

(21) Appl. No.: 15/563,773

(22) PCT Filed: Apr. 1, 2016
(86) PCT No.: PCT/US16/25493

§ 371 Date: Oct. 2, 2017

Related U.S. Application Data
(60) Provisional application No. 62/142,583, filed on Apr. 3, 2015.

(51) Int. Cl. G01N 27/414; G01N 33/487; G01N 33/551; G01N 33/573

(52) U.S. Cl. CPC G01N 27/4146; G01N 27/4145; G01N 33/48721; B82Y 15/00; G01N 33/573; G01N 2333/96433; G01N 33/551

(57) ABSTRACT

The present invention generally relates to nanoscale wires and other nanomaterials, including nanoscale wires used as sensors, including nanoscale wires comprising semiconductor nanowires, carbon nanotubes, graphene, or metal oxide nanomaterials. Certain aspects of the invention are generally directed to polymer coating on nanoscale wires that can be used to increase sensitivity to analytes, for example, in physiologically relevant conditions. For example, the polymer may have an average pore size comparable in size to an analyte. Accordingly, in some cases, the nanoscale wires can be used as sensors, even in ionic solutions, e.g., under physiologically relevant conditions. Other aspects of the invention include assays, sensors, kits, and/or other devices that include such nanoscale wires, methods of making and/or using such nanoscale wires, or the like.

### RELATED APPLICATIONS

This application claims the benefit of U.S. Provisional Patent Application Ser. No. 62/142,583, filed Apr. 3, 2015, entitled “Nanoscale Wires with Screening Polymers,” by Lieber, et al., incorporated herein by reference.

### GOVERNMENT FUNDING

This invention was made with government support under Grant No. 8DP1GM105379-05 awarded by the National Institutes of Health and Grant No. N00244-09-1-0078 awarded by the Department of Defense. The government has certain rights in the invention.

### FIELD

The present invention generally relates to nanoscale wires and other nanomaterials, including nanoscale wires used as sensors, including nanoscale wires comprising semiconductor nanowires, carbon nanotubes, graphene, or metal oxide nanomaterials.

### BACKGROUND

Interest in nanotechnology, including sub-microelectronic technologies such as semiconductor quantum dots and nanowires, has been motivated by the challenges of chemistry and physics at the nanoscale, and by the prospect of utilizing these structures in electronic and related devices. Nanoscopic articles might be well-suited for transport of charge carriers and excitons (e.g. electrons, electron pairs, etc.) and thus may be useful as building blocks in nanoscale electronics applications. Nanoscale wires are well-suited for efficient transport of charge carriers and excitons, and thus are expected to be important building blocks for nanoscale electronics.

Nanoscale materials have previously been suggested for use in various sensor applications. See, e.g., U.S. Pat. Nos. 7,129,554, 8,232,584, or 8,575,663; U.S. Pat. Apl. Pub. Nos. 2009/0299213, 2010/0087013, 2012/0267604, or 2014/0184196; or Int. Pat. Apl. Pub. Nos. WO 2013/166259 or WO 2014/043341; each incorporated herein by reference in its entirety. For example, different surface functionalizations of the nanoscale wire may permit interaction of the functionalized nanoscale wire with various entities, such as molecular entities, and the interaction induces a change in a property of the functionalized nanowire, which provides a mechanism for a nanoscale sensor device for detecting the presence or absence of an analyte suspected to be present in a sample. However, many difficulties remain in using nanoscale wires as sensors. For instance, it can be difficult to detect analytes in complex or “noisy” environments, such as in physiologically relevant conditions; for example, in serum, it can be very difficult to distinguish an analyte from other salts, ions, proteins, carbohydrates, etc. Thus, improvements in nanoscale sensor technologies are needed.

### SUMMARY

The present invention generally relates to nanoscale wires and other nanomaterials, including nanoscale wires used as sensors, including nanoscale wires comprising semiconductor nanowires, carbon nanotubes, graphene, or metal oxide nanomaterials. The subject matter of the present invention involves, in some cases, interrelated products, alternative solutions to a particular problem, and/or a plurality of different uses of one or more systems and/or articles.

In one aspect, the present invention is generally directed to an article. According to one set of embodiments, the article comprises a field-effect transistor comprising a gate comprising a nanoscale wire at least partially coated with a polymer having an average pore size of between about 2 nm and about 10 nm.

In another set of embodiments, the article comprises a field-effect transistor comprising a gate comprising a nanoscale wire. In some cases, the nanoscale wire is at least partially coated with polyethylene glycol.

The article, in accordance with yet another set of embodiments, includes an ionic solution comprising an analyte, and a field-effect transistor exposed to the ionic solution. In some embodiments, the field effect transistor comprises a gate comprising a nanoscale wire. In certain cases, the nanoscale wire at least partially coated with a polymer permeable to the analyte. The polymer may have an average pore size of between about 2 nm and about 10 nm.

In one embodiment, the article comprises a field-effect transistor comprising a gate comprising a carbon nanotube at least partially coated with a polymer, the polymer containing pores having an average diameter of between about 5 nm and about 50 nm. In another embodiment, the article comprises a field-effect transistor comprising a gate comprising a semiconductor metal oxide nanowire at least partially coated with a polymer, the polymer containing pores having an average diameter of between about 5 nm and about 50 nm. The article, in yet another embodiment, comprises a field-effect transistor comprising a gate comprising graphene at least partially coated with a polymer, the polymer containing pores having an average diameter of between about 5 nm and about 50 nm.

In one set of embodiments, the article includes a field-effect transistor exposed to an ionic solution, comprising nanoscale wire as a channel at least partially coated with a porous polymer layer having an average pore size of between 2 and about 10 nm, a source, a gate and a drain; a voltage generator apparatus capable of applying different bias voltages to the source and the gate; and a lock-in amplifier in electrical communication with the voltage generator and the drain.

In another set of embodiments, the article includes a field-effect transistor comprising graphene as a channel at least partially coated with a porous polymer layer having an average pore size of between 2 and about 10 nm, a source, a gate and a drain; a voltage generator apparatus capable of applying different bias voltages to the source and the gate; and a lock-in amplifier in electrical communication with the voltage generator and the drain.

In another aspect, the present invention is generally directed to a method. In one set of embodiments, the method includes exposing an analyte to a nanoscale wire at least partially coated with a polymer. In some cases, the polymer has an average pore size of at least the average size of the analyte and no more than 120% of the average size of the analyte. In some embodiments, the analyte has an average size of at least about 2 nm.

In another aspect, the present invention encompasses methods of making one or more of the embodiments described herein, for example, a nanoscale wire at least partially coated with a polymer. In some cases, the polymer may be porous, and/or the polymer may be permeable, e.g., to biomolecules or other analytes. In still another aspect, the present invention encompasses methods of using one or more of the embodiments described herein, for example, a nanoscale wire at least partially coated with a polymer.

In one set of embodiments, the method includes acts of providing a field-effect transistor device comprising a source, a drain, a gate and nanoscale wires, e.g., comprising graphene; modifying a porous polymer layer onto nanoscale wire or graphene surface, with average pore size in accordance with the size of analyte and no more than about 120% of the size of analyte; applying a bias voltage between source and drain; applying a bias voltage to the gate; and monitoring current flow between source and drain through preamplifiers.

In yet another embodiment, the method includes exposing a protein to a field-effect transistor comprising a gate comprising a carbon nanotube at least partially coated with a polymer, the polymer containing pores having an average diameter that is between 80% and 120% of the average diameter of the protein, whereby insertion of the protein into the pores alters the gating properties of the carbon nanotube. The method, in still another embodiment, includes exposing a protein to a field-effect transistor comprising a gate comprising graphene at least partially coated with a polymer, the polymer containing pores having an average diameter that is between 80% and 120% of the average diameter of the protein, whereby insertion of the protein into the pores alters the gating properties of the carbon nanotube. In another embodiment, the method includes exposing a protein to a field-effect transistor comprising a gate comprising a semiconductor metal oxide at least partially coated with a polymer, the polymer containing pores having an average diameter that is between 80% and 120% of the average diameter of the protein, whereby insertion of the protein into the pores alters the gating properties of the carbon nanotube.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B schematically illustrate porous polymers modified on a silicon nanowire field-effect transistor, and a sensing experimental set-up, in accordance with one embodiment of the invention;

FIGS. 2A-2D illustrate real-time PSA detection traces in various ionic strength buffer solutions, with and without modified porous polymer, in accordance with another embodiment of the invention;

FIGS. 3A-3B illustrate PSA detection performances in a relatively high ionic strength solution with porous polymer modified devices, in yet another embodiment of the invention;

FIGS. 4A-4C illustrate concentration-dependent real-time PSA detection trances and adsorption isothermals, in yet another embodiment of the invention;

FIG. 5 is a schematic diagram of a polymer coated on a nanoscale wire, in still another embodiment of the invention;

FIG. 6 illustrates a comparison of PSA detection in 10 mM PB for devices with and without polymer modification, in another embodiment of the invention; and

FIG. 7 illustrates a polymer coated on a nanoscale wire, in still another embodiment of the invention;

FIGS. 8A-8D illustrate real-time PSA detection traces in various ionic strength buffer solutions on graphene field effect sensors, in accordance with another embodiment of the invention; and

FIG. 9 illustrates modification ratio dependent PSA sensing signals on graphene field effect sensors, in yet another embodiment of the invention.

### DETAILED DESCRIPTION

The present invention generally relates to nanoscale wires and other nanomaterials, including nanoscale wires used as sensors, including nanoscale wires comprising semiconductor nanowires, carbon nanotubes, graphene, or metal oxide nanomaterials. Certain aspects of the invention are generally directed to polymer coating on nanoscale wires that can be used to increase sensitivity to analytes, for example, in physiologically relevant conditions. For example, the polymer may have an average pore size comparable in size to an analyte. Accordingly, in some cases, the nanoscale wires can be used as sensors, even in ionic solutions, e.g., under physiologically relevant conditions. Other aspects of the invention include assays, sensors, kits, and/or other devices that include such nanoscale wires, methods of making and/or using such nanoscale wires, or the like.

One aspect of the present invention is generally directed to a semiconductor at least partially coated with a polymer. The semiconductor may be, for instance, a nanoscale wire, and the semiconductor may be used as a sensor, e.g., as part of field-effect transistor (FET). In many ionic solutions (e.g., physiologically relevant solutions), the presence of ions may generally make it difficult for an analyte to interact with the nanoscale wire; this “screening” effect caused by the ionic solution may be quantified as a “Debye length,” as is known to those of ordinary skill in the art. However, in accordance with some embodiments of the invention, the presence of certain types of polymers on a nanoscale wire may effectively overcome Debye screening, partially or completely, which may improve sensitivity. In some cases, for example, the polymers may be porous, and/or the polymers may be permeable, e.g., to biomolecules or other analytes. For instance, the polymer may have an average pore size that is comparable in size to the analytes. Without wishing to be bound by any theory, it is believed that under such conditions, analyte is able to enter or otherwise interact with the polymer to affect the semiconductor, while preventing or reducing other ions or competing species from interacting with the semiconductor. The at least partial exclusion of ions from the semiconductor, e.g., due to the presence of the analyte as associated with the polymer, may thus serve to increase sensitivity of the semiconductor to the analyte.

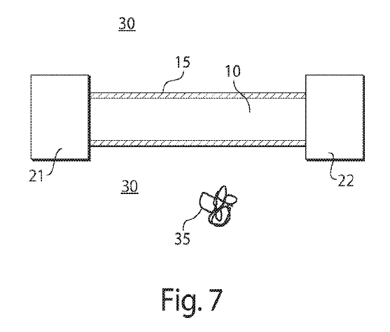

Turning now to FIG. 7, one non-limiting example embodiment of the invention is now discussed. In this figure, a nanoscale wire 10 is shown connecting two electrodes 21, 22. In some cases, these may represent a field-effect transistor (FET) where electrode 21 acts as a source, electrode 22 acts as a drain, and nanoscale wire 10 acts as a gate. The nanoscale wire may be a silicon nanowire, or other nanoscale wires as discussed below. On at least a portion of nanoscale wire 10 is a polymeric coating 15. In this figure, polymeric coating 15 surrounds nanoscale wire 10, but in other embodiments, only a portion of a nanoscale wire may be coated with a polymer.

One non-limiting example of such as polymer is polyethylene glycol; other examples are discussed below. Without wishing to be bound by any theory, it is believed that such polymers can act to at least partially screen ions. In some embodiments, the polymer may be porous and/or permeable to an analyte, for example, a biomolecule such as a protein or a peptide. For instance, the polymer have an average pore size similar in size to an analyte to be determined. In some cases, the analyte may be able to penetrate the polymer, and when interacting with the analyte, may prevent or reduce ions from entering.

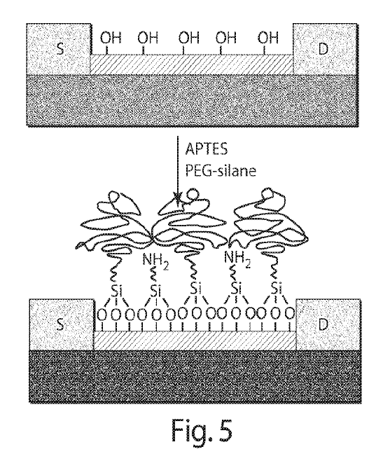

As will be discussed herein, a variety of techniques can be used to coat a polymer partially or completely on a nanoscale wire. The polymer may be covalently and/or non-covalently bound to at least a portion of the surface of the nanoscale wire. For example, in some cases, a nanoscale wire may be reacted with an oxysilane (such as 3-aminopropyl)triethoxysilane) to produce hydroxide (OH) groups, to which polymeric moieties (such as methoxyl silane polyethylene glycol) may be attached. See, for example, FIG. 5. As additional non-limiting examples, non-covalent bonds such as aromatic groups may be used to bond a polymer onto graphene, carbon nanotubes, organic semiconductors, etc., e.g., via pi-stacking or interactions between aromatic groups between the polymer and graphene, carbon nanotubes, organic semiconductors, or the like. In another example, a semiconductor may be coated using saline then followed by covalent bonds, e.g., using cross-linking reagents, or other nanoscale materials may be coated with aromatic precursors (such as pyrenebutyric acid) to allow covalent bonding with polymer by cross-linking reagents.

The polymer may be formed to have a certain average pore size that is comparable in size to an analyte to be determined.

An analyte 35 (for example, a protein such as prostate specific antigen or PSA) may be present in, for example, a fluid 30 (or other media) around the nanoscale wire. The presence of the analyte near the nanoscale wire may alter the electrical properties of the wire which may be determinable in some fashion. For example, the conductivity of the nanoscale wire may change as a result of the proximity of the analyte. In some cases, a reaction entity may be present immobilized relative to the nanoscale wire (e.g., attached to the nanoscale wire itself or to the polymer coating, etc.), which may facilitate the presence of the analyte near the nanoscale wire (e.g., by binding or attracting the analyte). However, it should be noted that the reaction entity is optional and not required in all embodiments.

The above discussion is a non-limiting example of certain embodiments of the present invention generally directed to a semiconductor nanoscale wire at least partially coated with a polymer. However, other embodiments are also possible. Accordingly, more generally, various aspects of the invention are directed to various systems and methods for making and using such semiconductor nanoscale wires.

In general, various aspects of the present invention are generally directed to a nanoscale wire able to interact with one or more analytes. The nanoscale wire may be, in some cases, a semiconductor nanoscale, comprising or consisting essentially of a semiconductor such as silicon or germanium. Other semiconductor or nanoscale materials are discussed below. The nanoscale wire may be doped or undoped. In some cases, the nanoscale wire is substantially cylindrical. In some cases, the nanoscale wire may have an average cross-sectional area of less than about 100 nm, less than about 50 nm, or less than about 30 nm, or other cross-sectional areas as described herein. In addition, in some embodiments a reaction entity may be present; the reaction entity may be, for example, an enzyme or an antibody. Other examples of reaction entities are discussed in more detail below.

In some aspects, the sensitivity of the nanoscale wire to the analyte may be enhanced by at least partially coating the nanoscale wire with a polymer. Non-limiting examples of polymers having relatively low dielectric constants include polyethylene glycol and polypropylene glycol. The polymers may have any suitable length and/or repeats. For instance, if polyethylene glycol, the polyethylene glycol may have an average molecular weight (weight average) of less than about 1,000, less than about 3,000, less than about 5,000, less than about 10,000, less than about 30,000, less than about 50,000, less than about 100,000, less than about 300,000, less than about 500,000, less than about 1,000,000, etc.

In some cases, the polymer may be relatively porous. For example, the polymer may be selected to a have an average pore size that is no more than about 105%, about 110%, about 115%, about 120%, about 125%, or about 130% of the average size of an analyte to be determined. In some cases, the average pore size is at least the size of the analyte. The average pore size may be uniform, or heterogeneous in some embodiments. In some cases, the average pore size of the polymer may be at least about 0.5 nm, at least about 1 nm, at least about 2 nm, at least about 3 nm, at least about 4 nm, at least about 5 nm, at least about 6 nm, at least about 7 nm, at least about 8 nm, at least about 9 nm, at least about 10 nm, at least about 15 nm, at least about 20 nm, at least about 25 nm, at least about 30 nm, at least about 40 nm, at least about 50 nm, etc. In some cases, the average pore size of the polymer may be no more than about 50 nm, no more than about 40 nm, no more than about 30 nm, no more than about 25 nm, no more than about 20 nm, no more than about 15 nm, no more than about 10 nm, no more than about 9 nm, no more than about 8 nm, no more than about 7 nm, no more than about 6 nm, no more than about 5 nm, no more than about 3 nm, no more than about 2 nm, no more than about 1 nm, no more than about 0.5 nm, etc. Combinations of any of these are also possible. For example, the average pore size may be between about 2 nm and about 10 nm, between about 2 nm and about 8 nm, between about 2 nm and about 5 nm, between 5 nm and about 8 nm, between 5 nm and about 10 nm, between 7 nm and about 10 nm, or the like.

In another set of embodiments, the polymer may have an average pore size able to admit a protein (e.g., a globular or generally spherical protein) or other biomolecule having a molecular weight of at least about 5 Da, at least about 10 Da, at least about 30 Da, at least about 50 Da, at least about 100 Da, at least about 300 Da, at least about 500 Da, at least about 1 kDa, at least about 3 kDa, at least about 5 kDa, at least about 10 kDa, at least about 30 kDa, at least about 50 kDa, at least about 100 kDa, at least about 300 kDa, at least about 500 kDa, at least about 100 kDa, at least about 300 kDa, at least about 500 kDa, at least about 1000 kDa, at least about 3,000 kDa, at least about 5,000 kDa, and/or no more than 5,000 kDa, no more than about 3,000 kDa, no more than about 1,000 kDa, no more than about 500 kDa, no more than about 300 kDa, no more than about 100 kDa, no more than about 50 kDa, no more than about 30 kDa, no more than about 10 kDa, no more than about 5 kDa, no more than about 3 kDa, no more than about 1 kDa, no more than about 500 Da, no more than about 300 Da, no more than about 100 Da, no more than about 50 Da, no more than about 30 Da, no more than about 10 Da, no more than about 5 Da, etc. Combinations of these are also possible, e.g., the polymer may have an average pore size able to admit a protein of between about 100 Da and about 500 kDa.

The polymer may be relatively hydrophilic or relatively hydrophobic. The average pore size can be determined, for example, using techniques such as SEM, AFM, or X-ray microscopy.

In some cases, the average pore size of the polymer may be controlled by controlling the ratio of smaller molecules (e.g., which are unable to participate in the polymerization reaction, or which may be bound to the nanoscale wire surface although they are not part of the polymer in some cases) to polymer when the polymer is formed or attached to the nanoscale wires. For example, if the polymer is PEG-silane, ethylene glycol may be mixed with APTES ((3-aminopropyl)triethoxysilane), which can exit the polymer after the polymer has formed, thereby leaving pores within the polymer. By simply controlling the ratio of smaller molecules to polymer, the size of the pores can be readily controlled. The exact ratio may depend on the types of polymers used and the analyte to be determined.

The polymer may fully or partially coat the nanoscale wire. For example, the polymer may coat at least about 50%, at least about 70%, at least about 90%, or substantially all of the surface of the nanoscale wire. In addition, in some cases, the polymer may be relatively porous or permeable to an analyte, for example, a biomolecule such as a protein or a peptide. In some cases, the polymer may be exposed to the nanoscale wire under conditions such that the polymer does not form a homogenous monolayer on the nanoscale wire, e.g., such that the polymer is somewhat porous around the nanoscale wire.

In some aspects, the nanoscale wire may be used to determine an analyte, e.g., contained in solution. The analyte may be, for example, a protein, a peptide, a virus, an enzyme, a nucleic acid (e.g., DNA or RNA), a chemical compound, an ion, or the like and may be, for instance, dissolved or suspended in solution. The analyte may be determined, for instance, to determine or diagnose cancer or other medical conditions (e.g., by determining a suitable analyte and diagnosing the medical condition based on the determination of the analyte), to determine a suitable drug (e.g., as part of a drug assay or a drug screen, for example, to identify a drug able to treat a medical condition such as cancer or aging), to determine toxins or other environmental agents (e.g., by determining binding of the toxin to a receptor), or the like.

“Determine,” in contexts such as this, generally refers to the analysis of a species, for example, quantitatively or qualitatively, and/or the detection of the presence or absence of the species. “Determining” may also refer to the analysis of an interaction between two or more species in certain contexts, for example, quantitatively or qualitatively, and/or by detecting the presence or absence of the interaction, e.g. determination of the binding between two species.

The nanoscale wire, in some embodiments, may be responsive to a property external of the nanoscale wire, e.g., a chemical property, an electrical property, a physical property, etc. Such determination may be qualitative and/or quantitative. For example, in one set of embodiments, the nanoscale wire may be responsive to a chemical property of the environment surrounding the nanoscale wire, e.g., due to an analyte. For example, an electrical property of the nanoscale wire can be affected by a chemical environment surrounding the nanoscale wire, and the electrical property can be thereby determined to determine the chemical environment surrounding the nanoscale wire. Further non-limiting examples of such nanoscale wires are discussed in U.S. Pat. No. 7,129,554, filed Oct. 31, 2006, entitled “Nanosensors,” by Lieber, et al., incorporated herein by reference in its entirety.

As a non-limiting example, the nanoscale wire may have the ability to bind to an analyte indicative of a chemical property of the environment surrounding the nanoscale wire (e.g., hydrogen ions for pH, or concentration for another analyte of interest, such as a protein). In some cases, the nanoscale wire may be partially or fully functionalized, i.e. comprising surface functional moieties, to which an analyte is able to bind, thereby causing a determinable property change to the nanoscale wire, e.g., a change to the resistivity or impedance of the nanoscale wire. In some cases, a reaction entity may be present. The binding of the analyte can be specific or non-specific. Functional moieties may include simple groups, selected from the groups including, but not limited to, —OH, —CHO, —COOH, —SO₃H, —CN, —NH₂, —SH, —COSH, —COOR, halide; biomolecular entities including, but not limited to, amino acids, proteins, sugars, DNA, antibodies, antigens, and enzymes; polymer chains (e.g., having chain lengths less than the diameter of the nanowire core), a shell of material comprising, for example, metals, semiconductors, and insulators, which may be a metallic element, an oxide, an sulfide, a nitride, a selenide, a polymer and a polymer gel. Examples of polymers include, but are not limited to, polyamide, polyester, polyimide, or polyacrylic polymers, or other polymers as discussed herein.

In another aspect, the present invention generally relates to the attachment of entities, such as polymers or reaction entities, to the surfaces of nanoscale wires, in some cases by using covalent or non-covalent bonding. The entity is thus immobilized with respect to the surface of the nanoscale wire. In some embodiments, a linker is used to covalently immobilize the entity with respect to the nanoscale wire. For example, a nanoscale wire may have a core, and optionally a shell, and a linker may covalently immobilize the entity with respect to the nanoscale wire. In some cases, the entity may be covalently immobilized onto the surface of the nanoscale wire and/or immobilized at relatively short distances, depending on the size of the linker and/or the precursors thereof. For instance, the entity may be immobilized at a distance of less than about 20 nm, less than about 15 nm, less than about 10 nm, less than about 9 nm, less than about 8 nm, less than about 7 nm, less than about 6 nm, less than about 5 nm, less than about 4 nm, less than about 3 nm, less than about 2 nm, or less than about 1 nm from the surface of the nanoscale wire. In some cases, the proximity of the entity may control or otherwise affect electronic and/or other properties of the nanoscale wire, for example, the conductivity of the nanoscale wire, e.g., as discussed herein.

Non-limiting examples of techniques of attaching entities to nanoscale wires include those described herein. For instance, certain embodiments provide for the attaching, to the surface of a nanoscale wire, of a linker or a precursor of a molecule such as a linker to the nanoscale wire, which may be used to add, e.g., a polymer or a reaction entity to the surface of the nanoscale wire. By using such techniques, the distance between the entity and the nanoscale wire may be controlled. For example, the distance may be controlled to be less than about 20 nm, less than about 15 nm, less than about 10 nm, less than about 5 nm, etc. In some cases, the remaining precursor molecules on the surface of the nanoscale wire, which are not bound to a suitable entity, may be passivated. For instance, the remaining precursor molecules may be passivated by exposure of the nanoscale wire to a passivating agent such as ethanolamine or Si₃N₄.

Non-limiting examples of chemistries suitable for attaching entities to surfaces of nanoscale wires, optionally via one or more linkers, include the following. In one set of embodiments of the present invention, the surface of the nanoscale wire may be functionalized, for example, the surface may be functionalized with aldehydes, amines, thiols, or the like, which may form nitrogen-containing or sulfur-containing covalent bonds. For instance, in some embodiments, the entity may be covalently bound to the nanoscale wire through the use of a moiety such as an aldehyde moiety, an amine moiety, and/or a thiol moiety.

In certain embodiments, a nanoscale wire may be reacted with an aldehyde, amine, or a thiol in solution to functionalize the nanoscale wire with the appropriate moiety, e.g., such that the surface of the nanoscale wire includes terminal aldehyde, amine, and/or thiol groups. For example, the solution may contain an aldehyde such as aldehyde propyltrimethoxysilane ((CH₃O)₃SiCH₂CH₂CHO), or other aldehydes, for example, having a formula (OCHR¹)(R²O)(R³O)(R⁴O)Si, where each R is independently an alkyl or other carbon-containing moiety. All, or only a portion of, the surface of the nanoscale wire may be functionalized with aldehyde moieties (for example, a portion of the nanoscale wire may be blocked or shielded, prior to aldehydization of the surface).

Examples of suitable amines or thiols include amino- and thiol-functionalized silane derivatives, for instance, (3-aminopropyl)triethoxysilane (APTES) or trimethoxy propylamine silane ((CH₃O)₃SiCH₂CH₂CH₂NH₂) or propylthiol trimethoxy silane ((CH₃O)₃SiCH₂CH₂CH₂SH), which may react with all, or only a portion of, the surface of the nanoscale wire to form, surfaces functionalized with, respectively, amines or thiols. Thus, for example, a polymeric silane, such as PEG-silane, may be reacted with amine groups to immobilize the polymer to at least a portion of the surface of the nanoscale wire, e.g., as is shown in the example of FIG. 5.

Other potentially suitable amines may have a formula (Z¹Z²NR¹)(R²O)(R³O)(R⁴O)Si, where each R is independently an alkyl or other carbon-containing moiety and each Z independently is —H or an alkyl or other carbon-containing moiety; other potentially suitable thiols may have a formula (HSR¹)(R²O)(R³O)(R⁴O)Si. In some cases, the derivative may have more than one functional group, for example, the derivative may have an amine and a thiol group, an amine and an aldehyde group, a thiol and an aldehyde group, etc.

One or more entities such as polymers, proteins, enzymes, nucleic acids, antibodies, receptors, ligands, etc., may then be reacted with the aldehyde, amine, and/or thiol moieties to covalently bind the entity to the nanoscale wire. In some cases, after the entity has been fastened to the nanoscale wire, the surface of the nanoscale wire, including any unreacted moieties, is then passivated, e.g., blocked with one or more compounds that causes the moieties to become unreactive. Non-limiting examples of such passivating agents include ethanolamine. For example, a solution may be added to the nanowires that includes one or more passivating agents.

In some cases, the entity covalently binds to an aldehyde group via a reaction between a functional group of the entity and the aldehyde. For instance, if the entity contains a primary amine (RNH₂), the primary amine can react with the aldehyde to produce an imine bond (R¹CH═NR²), e.g. as in a reaction:

R¹CHO+R²NH₂ [symbol] R¹CH═NR²+H₂O,

where R¹ represents the surface of the nanoscale wire and/or a species immobilized relative to the surface of the nanoscale wire, and R² is an alkyl or other carbon-containing moiety. Thus, as a particular, non-limiting example, an entity containing an amine, such as a polymer, a protein, an antibody, or an enzyme, may be reacted with an aldehyde on the surface of the nanoscale wire, thereby covalently binding the entity to the surface of the nanoscale wire. As another non-limiting example, an entity that does not contain a primary amine, such as a nucleic acid molecule, may be modified to include a primary amine, and then the primary amine reacted with an aldehyde on the surface of the nanoscale wire, thereby binding the entity to the surface of the nanoscale wire. Similarly, the entity may covalently bind to an amine or a thiol group via reaction between a functional group of the entity and an amine or thiol.

In one set of embodiments, a nitrogen-containing covalent bond may be formed between an entity and a functional group present on the surface of a nanoscale wire, thereby immobilizing the entity with respect to the surface of the nanoscale wire. In one embodiment, the covalent bond is a nitrogen-containing covalent bond, such as an imine bond, an amide bond, a carbamate bond, etc. As an example, an entity containing an amine may react with an aldehyde present on the surface of a nanoscale wire to form an imine bond, e.g., as previously described. As another example, an entity containing an aldehyde may react with an amine via the following reaction:

R¹NH₂+R²CHO [symbol] R¹N═CHR²+H₂O,

where R¹ represents the surface of the nanoscale wire and/or a species immobilized relative to the surface of the nanoscale wire, and R² is an alkyl or other carbon-containing moiety. In this reaction, an imine bond is produced, immobilizing R² with respect to R¹. As yet another example, an amine may react with a carboxylic acid, producing an amide bond immobilizing an entity with respect to a surface, e.g., as follows:
or

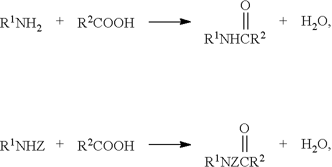

where R¹ represents the surface of the nanoscale wire and/or a species covalently or non-covalently immobilized relative to the surface of the nanoscale wire, R² is an alkyl or other carbon-containing moiety, and Z is —H or an alkyl or other carbon-containing moiety. For instance, an entity comprising an N-hydroxysuccinimide group or other similar leaving group may react with an amine to form a carbamate bond, e.g., as in a reaction:

R¹NH₂+R²COO—(NHS) [symbol] R¹NHCOOR².

where R¹ represents the surface of the nanoscale wire and/or a species immobilized relative to the surface of the nanoscale wire, R² is an alkyl or other carbon-containing moiety, and (NHS) is an N-hydroxysuccinimide group. In another set of embodiments, R¹ and R² mentioned above may be exchanged to each other, as an alkyl or other carbon-containing moiety, and species immobilized to the surface of nanoscale wire, respectively. Other examples of reactions include reactions forming carbamate (R¹R²N—C(═O)—O—R³) bonds.

In another example, a sulfur-containing covalent bond may be formed between an entity (e.g., a polymer or a reaction entity) and a functional group present on the surface of a nanoscale wire, thereby immobilizing the entity with respect to the surface of the nanoscale wire. An example is a reaction involving a thiol. For instance, in one embodiment, an entity may be covalently immobilized to a thiol group via the following reaction:

R¹SH+R²-Maleimide [symbol] R¹S-Maleimide-R²,

where R¹ represents the surface of the nanoscale wire and/or a species immobilized relative to the surface of the nanoscale wire, and R² is an alkyl or other carbon-containing moiety. Thus, R² is immobilized with respect to R¹ via a sulfur-containing covalent bond.

In some but not all embodiments, the nanoscale wire has a reaction entity able to interact with or determine an analyte. Nanoscale sensing elements of the invention may be used, for example, to determine pH or metal ions, viruses, proteins or enzymes, nucleic acids (e.g. DNA, RNA, PNA, etc.), drugs, sugars, carbohydrates, a toxin (e.g., a harmful chemical, such as a chemical produced by a living organism that is harmful to other organisms), small molecules (e.g., having molecular weights of less than about 2000 Da, less than about 1500 Da, or less than about 1000 Da), or other analytes, as further described herein. The analyte may be charged, or uncharged in some embodiments. In certain embodiments, single entities may be determined, for example, a single virus, a single protein, a single enzyme, a single nucleic acid molecule, a single drug molecule, a single carbohydrate molecule, etc. In some cases, the sensing element includes a detector constructed and arranged to determine a change in a property of the nanoscale wire, for example, a change in an electrical property of the nanoscale wire, such as voltage, current, conductivity, resistivity, inductance, impedance, electrical change, an electromagnetic change, etc., e.g., as discussed herein.

As used herein, the term “reaction entity” refers to any entity that can interact with an analyte in such a manner as to cause a detectable change in a property of a nanoscale wire. The reaction entity may comprise a binding partner to which the analyte binds. The reaction entity, when a binding partner, can comprise a specific binding partner of the analyte, e.g., specifically or non-specifically. In some cases, the reaction entity can form a partial or complete coating on the nanoscale wire. Non-limiting examples of reaction entities include a nucleic acid (e.g., DNA or RNA), an antibody, a sugar or a carbohydrate, a protein or an enzyme, a ganglioside or a surfactant, etc., e.g., as discussed herein.

In one set of embodiments, a reaction entity associated with the nanoscale wire is able to interact with an analyte. The reaction entity, as associated with or immobilized relative to the nanoscale wire, may be positioned in relation to the nanoscale wire (e.g., in close proximity or in contact) such that the analyte can be determined by determining a change in a characteristic or property of the nanoscale wire. Interaction of the analyte with the reaction entity may cause a detectable change or modulation in a property of the nanoscale wire, for example, through electrical coupling with the reaction entity. The term “electrically coupled” or “electrocoupling,” when used with reference to a nanoscale wire and an analyte, or other moiety such as a reaction entity, refers to an association between any of the analyte, other moiety, and the nanoscale wire such that electrons can move from one to the other, or in which a change in an electrical characteristic of one can be determined by the other. This can include electron flow between these entities, or a change in a state of charge, oxidation, or the like, that can be determined by the nanoscale wire. As examples, electrical coupling or immobilization can include direct covalent linkage between the analyte or other moiety and the nanoscale wire, indirect covalent coupling (for instance, via a linker, and/or a plurality of linkers, e.g., serially), direct or indirect ionic bonding between the analyte (or other moiety) and the nanoscale wire, direct or indirect bonding of both the analyte and the nanoscale wire to a particle (i.e., the particle acts as a linker between the analyte and the nanoscale wire), direct or indirect bonding of both the analyte and the nanoscale wire to a common surface (i.e., the surface acts as a linker), or other types of bonding or interactions (e.g. hydrophobic interactions or hydrogen bonding). In some cases, no actual covalent bonding is required; for example, the analyte or other moiety may simply be contacted or be in proximity with the nanoscale wire. There also need not necessarily be any contact between the nanoscale wire and the analyte or other moiety, for example, due to electric field effects, or where the nanoscale wire is sufficiently close to the analyte to permit electron tunneling between the analyte and the nanoscale wire.

Thus, the reaction entity may be positioned relative to the nanoscale wire to cause a detectable change in the nanoscale wire. In some cases, the reaction entity may be positioned within about 100 nm of the nanoscale wire, within about 75 nm of the nanoscale wire, within about 50 nm of the nanoscale wire, within about 20 nm of the nanoscale wire, within about 15 nm of the nanoscale wire, or within about 10 nm of the nanoscale wire. In some cases, the reaction entity is positioned less than about 5 nm from the nanoscale wire. In other cases, the reaction entity is positioned within about 4 nm, within about 3 nm, within about 2 nm, or within about 1 nm of the nanoscale wire.

In some embodiments, the reaction entity is fastened to or directly bonded (e.g., covalently) to the nanoscale wire, e.g., as further described herein. However, in other embodiments, the reaction entity is not directly bonded to the nanoscale wire, but is otherwise immobilized relative to the nanoscale wire, i.e., the reaction entity is indirectly immobilized relative to the nanoscale wire. For instance, the reaction entity may be attached to the nanoscale wire through a linker, i.e., a species (or plurality of species) to which the reaction entity and the nanoscale wire are each immobilized relative thereto, e.g., covalently or non-covalently bound to. As an example, a linker may be directly bonded to the nanoscale wire, and the reaction entity may be directly bonded to the linker, or the reaction entity may not be directly bonded to the linker, but immobilized relative to the linker, e.g., through the use of non-covalent bonds such as hydrogen bonding (e.g., as in complementary nucleic acid-nucleic acid interactions), hydrophobic interactions (e.g., between hydrocarbon chains), entropic interactions, or the like. The linker may or may not be directly bonded (e.g., covalently) to the nanoscale wire.

Thus, the reaction may be attached to the nanoscale wire using various techniques, such as covalent or non-covalent bonding. The entity is thus immobilized with respect to the surface of the nanoscale wire. The reaction entity may be directly immobilized to the nanoscale wire, or in some embodiments, a linker is used to covalently immobilize the entity with respect to the nanoscale wire. In some cases, the entity may be covalently immobilized with respect to the surface of the nanoscale wire at relatively short distances, depending on the size of the linker and/or the precursors thereof. For instance, the entity may be immobilized at a distance of less than about 20 nm, less than about 15 nm, less than about 10 nm, less than about 9 nm, less than about 8 nm, less than about 7 nm, less than about 6 nm, less than about 5 nm, less than about 4 nm, less than about 3 nm, less than about 2 nm, or less than about 1 nm from the surface of the nanoscale wire. In some cases, the proximity of the entity may control or otherwise affect electronic and/or other properties of the nanoscale wire, for example, the conductivity of the nanoscale wire.

Non-limiting examples of chemistries suitable for attaching entities to surfaces of nanoscale wires, optionally via one or more linkers, include the following. In one set of embodiments of the present invention, the surface of the nanoscale wire may be functionalized, for example, the surface may be functionalized with aldehydes, amines, thiols, carboxyls, or the like, which may form nitrogen-containing or sulfur-containing covalent bonds. For instance, in some embodiments, the reaction entity may be covalently bound to the nanoscale wire through the use of a moiety such as an aldehyde moiety, an amine moiety, a carboxyl moiety, and/or a thiol moiety. In certain embodiments, a nanoscale wire may be reacted with an aldehyde, amine, carboxyl, or a thiol in solution to functionalize the nanoscale wire with the appropriate moiety, e.g., such that the surface of the nanoscale wire includes terminal aldehyde, amine, and/or thiol groups. Additional examples are disclosed in U.S. patent application Ser. No. 11/501,466, filed Aug. 9, 2006, entitled “Nanoscale Sensors,” by Lieber, et al., incorporated herein by reference.

One or more entities, e.g., reaction entities such as proteins, enzymes, nucleic acids, antibodies, receptors, ligands, etc., may then be reacted with the aldehyde, amine, carboxyl, and/or thiol moieties to covalently bind or link the entity to the nanoscale wire. In some cases, after the entity has been fastened to the nanoscale wire, the surface of the nanoscale wire, including any unreacted moieties, is then passivated, e.g., blocked with one or more compounds that causes the moieties to become unreactive. Non-limiting examples of such passivating agents include ethanolamine. For example, a solution may be added to the nanowires that includes one or more passivating agents.

The binding between an analyte and a reaction entity may be specific or non-specific. The term “binding” refers to the interaction between a corresponding pair of molecules or surfaces that exhibit mutual affinity or binding capacity, typically due to specific or non-specific binding or interaction, including, but not limited to, biochemical, physiological, and/or chemical interactions. “Biological binding” defines a type of interaction that occurs between pairs of molecules including proteins, nucleic acids, glycoproteins, carbohydrates, hormones and the like. Specific non-limiting examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, virus/cell surface receptor, etc.

The term “binding partner” refers to a molecule that can undergo binding with a particular molecule. Biological binding partners are examples. For example, Protein A is a binding partner of the biological molecule IgG, and vice versa. Other non-limiting examples include nucleic acid-nucleic acid binding, nucleic acid-protein binding, protein-protein binding, enzyme-substrate binding, receptor-ligand binding, receptor-hormone binding, antibody-antigen binding, etc. Binding partners include specific, semi-specific, and non-specific binding partners as known to those of ordinary skill in the art. Examples of specific binding include, but are not limited to, receptor-ligand interactions, enzyme-substrate interactions, nucleic acid-complementary nucleic acid interactions, antibody-antigen interactions, or the like. The binding may be by one or more of a variety of mechanisms including, but not limited to, ionic interactions, covalent interactions, hydrophobic interactions, van der Waals interactions, etc.

As mentioned, certain aspects of the present invention include a nanoscale wire (or nanoscopic wire) or other nanostructured or nanoscale material comprising one or more semiconductors, graphene, etc., as discussed herein. In some cases, the nanoscale wire is doped. The nanoscale wire may be, for example, a nanorod, a nanowire, a nanowhisker, or a nanotube. The nanoscale wire may be used in a device, for example, as a semiconductor component, a pathway, etc. The criteria for selection of nanoscale wires and other conductors or semiconductors may be based, in some instances, upon whether the nanoscale wire is able to interact with an analyte, and/or whether the appropriate reaction entity, e.g. a binding partner, can be easily attached to the surface of the nanoscale wire, or the appropriate reaction entity, e.g. a binding partner, is near the surface of the nanoscale wire.

Thus, certain embodiments of the invention are generally directed to nanoscale wires, including nanotubes and nanowires. In some embodiments, however, the invention comprises articles that may include wires of greater than nanometer size (e.g., micrometer-sized). As used herein, “nanoscopic-scale,” “nanoscopic,” “nanometer-scale,” “nanoscale,” the “nano-” prefix (for example, as in “nanostructured”), and the like generally refers to elements or articles having widths or diameters of less than about 1 micron, and less than about 100 nm in some cases. Specified widths can be a smallest width (i.e. a width as specified where, at that location, the article can have a larger width in a different dimension), or a largest width (i.e. where, at that location, the article has a width that is no wider than as specified, but can have a length that is greater).

The nanoscale wire may be used to connect two electronic components such that they are in electronic communication with each other. It may be a semiconductor nanowire, or have a conductivity (or resistivity) of or of similar magnitude to any semiconductor or any metal. For instance, the nanoscale wire may have resistivities of less than about 100 microOhm cm (μΩ cm). In some cases, the nanoscale wire will have a resistivity lower than about 10⁻³ ohm meters, lower than about 10⁻⁴ ohm meters, or lower than about 10⁻⁶ ohm meters or 10⁻⁷ ohm meters. The semiconductor may be an element (or group of elements) having semiconductive or semi-metallic properties (i.e., between metallic and non-metallic properties). An example of a semiconductor is silicon. Other non-limiting examples include gallium, germanium, diamond (carbon), tin, selenium, tellurium, boron, or phosphorous.

A “nanoscale wire” (also known herein as a “nanoscopic-scale wire” or “nanoscopic wire”) generally is a wire, that at any point along its length, has at least one cross-sectional dimension and, in some embodiments, two orthogonal cross-sectional dimensions less than 1 micron, less than about 500 nm, less than about 300 nm, less than about 200 nm, less than about 150 nm, less than about 100 nm, less than about 70, less than about 50 nm, less than about 30 nm, less than about 20 nm, less than about 10 nm, or less than about 5 nm. In other embodiments, the cross-sectional dimension can be less than 2 nm or 1 nm. The cross-section of a nanoscale wire may be of any arbitrary shape, including, but not limited to, circular, square, rectangular, annular, polygonal, or elliptical, and may be a regular or an irregular shape. The nanoscale wire may be solid or hollow.

A non-limiting list of examples of materials from which nanoscale wires of the invention can be made appears below. Any nanoscale wire can be used in any of the embodiments described herein, including carbon nanotubes, molecular wires (i.e., wires formed of a single molecule), nanorods, nanowires, nanowhiskers, organic or inorganic conductive or semiconducting polymers, and the like. Other conductive or semiconducting elements that may not be molecular wires, but are of various small nanoscopic-scale dimensions, can also be used in some instances, e.g. inorganic structures such as main group and metal atom-based wire-like silicon, transition metal-containing wires, gallium arsenide, gallium nitride, indium phosphide, germanium, cadmium selenide, etc., graphene materials, 2D semiconductors, or the like.

The nanoscale wires, in some cases, may be formed having dimensions or lengths of at least about 1 micron, at least about 3 microns, at least about 5 microns, or at least about 10 microns or about 20 microns in length. In some cases, however, the length of the nanoscale wire may be less than about 100 nm, less than about 80 nm, less than about 60 nm, less than about 40 nm, less than about 20 nm, less than about 10 nm, or less than about 5 nm. The nanoscale wires may be an elongated nanoscale wire, or have an aspect ratio (length to thickness) of greater than about 2:1, greater than about 3:1, greater than about 4:1, greater than about 5:1, greater than about 10:1, greater than about 25:1, greater than about 50:1, greater than about 75:1, greater than about 100:1, greater than about 150:1, greater than about 250:1, greater than about 500:1, greater than about 750:1, or greater than about 1000:1 or more in some cases.

The nanoscale wire may, in certain embodiments, be a nanowire or a nanotube. A “nanowire” (e.g. comprising silicon and/or another semiconductor material) is a nanoscale wire that is typically a solid (i.e., not hollow) wire. A “non-nanotube nanowire” is any nanowire that is not a nanotube. A “nanotube” is a nanoscale wire that is hollow, or that has a hollowed-out core.

Many nanoscale wire as used in accordance with the present invention are individual nanoscale wires. As used herein, “individual nanoscale wire” means a nanoscale wire free of contact with another nanoscale wire (but not excluding contact of a type that may be desired between individual nanoscale wires, e.g., as in a crossbar array). For example, an “individual” or a “free-standing” article may, at some point in its life, not be attached to another article, for example, with another nanoscale wire, or the free-standing article may be in solution. An “individual” or a “free-standing” article is one that can be (but need not be) removed from the location where it is made, as an individual article, and transported to a different location and combined with different components to make a functional device such as those described herein.

In another set of embodiments, the nanoscale wire (or other nanostructured material) may include additional materials, such as semiconductor materials, dopants, organic compounds, inorganic compounds, etc. The following are non-limiting examples of materials that may be used as dopants within the nanoscale wire. The dopant may be an elemental semiconductor, for example, silicon, germanium, tin, selenium, tellurium, boron, diamond, or phosphorous. The dopant may also be a solid solution of various elemental semiconductors. Examples include a mixture of boron and carbon, a mixture of boron and P(BP₆), a mixture of boron and silicon, a mixture of silicon and carbon, a mixture of silicon and germanium, a mixture of silicon and tin, a mixture of germanium and tin, etc. In some embodiments, the dopant may include mixtures of Group IV elements, for example, a mixture of silicon and carbon, or a mixture of silicon and germanium. In other embodiments, the dopant may include mixtures of Group III and Group V elements, for example, BN, BP, BAs, AlN, AlP, AlAs, AlSb, GaN, GaP, GaAs, GaSb, InN, InP, InAs, or InSb. Mixtures of these combinations may also be used, for example, a mixture of BN/BP/BAs, or BN/AlP. In other embodiments, the dopants may include mixtures of Group III and Group V elements. For example, the mixtures may include AlGaN, GaPAs, InPAs, GaInN, AlGaInN, GaInAsP, or the like. In other embodiments, the dopants may also include mixtures of Group II and Group VI elements. For example, the dopant may include mixtures of ZnO, ZnS, ZnSe, ZnTe, CdS, CdSe, CdTe, HgS, HgSe, HgTe, BeS, BeSe, BeTe, MgS, MgSe, or the like. Alloys or mixtures of these dopants are also possible, for example, ZnCdSe, or ZnSSe or the like. Additionally, mixtures of different groups of semiconductors may also be possible, for example, combinations of Group II-Group VI and Group III-Group V elements, such as (GaAs)ₓ(ZnS)₁₋ₓ. Other non-limiting examples of dopants may include mixtures of Group IV and Group VI elements, for example GeS, GeSe, GeTe, SnS, SnSe, SnTe, PbO, PbS, PbSe, PbTe, etc. Other dopant mixtures may include mixtures of Group I elements and Group VII elements, such as CuF, CuCl, CuBr, CuI, AgF, AgCl, AgBr, AgI, or the like. Other dopant mixtures may include different mixtures of these elements, such as BeSiN₂, CaCN₂, ZnGeP₂, CdSnAs₂, ZnSnSb₂, CuGeP₃, CuSi₂P₃, Si₃N₄, Ge₃N₄, Al₂O₃, (Al, Ga, In)₂(S, Se, Te)₃, Al₂CO, (Cu, Ag)(Al, Ga, In, Tl, Fe)(S, Se, Te)₂ or the like.

As a non-limiting example, a p-type dopant may be selected from Group III, and an n-type dopant may be selected from Group V. For instance, a p-type dopant may include at least one of B, Al and In, and an n-type dopant may include at least one of P, As and Sb. For Group III-Group V mixtures, a p-type dopant may be selected from Group II, including one or more of Mg, Zn, Cd and Hg, or Group IV, including one or more of C and Si. An n-type dopant may be selected from at least one of Si, Ge, Sn, S, Se and Te. It will be understood that nanoscale wires are not limited to these dopants, but may include other elements, alloys, or mixtures as well.

As used herein, the term “Group,” with reference to the Periodic Table, is given its usual definition as understood by one of ordinary skill in the art. For instance, the Group II elements include Mg and Ca, as well as the Group II transition elements, such as Zn, Cd, and Hg. Similarly, the Group III elements include B, Al, Ga, In and Tl; the Group IV elements include C, Si, Ge, Sn, and Pb; the Group V elements include N, P, As, Sb and Bi; and the Group VI elements include O, S, Se, Te and Po. Combinations involving more than one element from each Group are also possible. For example, a Group II-VI material may include at least one element from Group II and at least one element from Group VI, e.g., ZnS, ZnSe, ZnSSe, ZnCdS, CdS, or CdSe. Similarly, a Group III-V material may include at least one element from Group III and at least one element from Group V, for example GaAs, GaP, GaAsP, InAs, InP, AlGaAs, or InAsP. Other dopants may also be included with these materials and combinations thereof, for example, transition metals such as Fe, Co, Te, Au, and the like. The nanoscale wire may further include, in some cases, any organic or inorganic molecules. In some cases, the organic or inorganic molecules are polarizable and/or have multiple charge states.

In some embodiments, at least a portion of a nanoscale wire may be a bulk-doped semiconductor. As used herein, a “bulk-doped” article (e. g. an article, or a section or region of an article) is an article for which a dopant is incorporated substantially throughout the crystalline lattice of the article, as opposed to an article in which a dopant is only incorporated in particular regions of the crystal lattice at the atomic scale, for example, only on the surface or exterior. For example, some articles are typically doped after the base material is grown, and thus the dopant only extends a finite distance from the surface or exterior into the interior of the crystalline lattice. It should be understood that “bulk-doped” does not define or reflect a concentration or amount of doping in a semiconductor, nor does it necessarily indicate that the doping is uniform. In particular, in some embodiments, a bulk-doped semiconductor may comprise two or more bulk-doped regions. Thus, as used herein to describe nanoscale wire, “doped” refers to bulk-doped nanoscale wires. “Heavily doped” and “lightly doped” are terms the meanings of which are understood by those of ordinary skill in the art.

In one set of embodiments, the invention includes a nanoscale wire (or other nanostructured material) that is a single crystal. As used herein, a “single crystal” item (e.g., a semiconductor) is an item that has covalent bonding, ionic bonding, or a combination thereof throughout the item. Such a single-crystal item may include defects in the crystal, but is to be distinguished from an item that includes one or more crystals, not ionically or covalently bonded, but merely in close proximity to one another.

In yet another set of embodiments, the nanoscale wire (or other nanostructured material) may comprise two or more regions having different compositions. Each region of the nanoscale wire may have any shape or dimension, and these can be the same or different between regions. For example, a region may have a smallest dimension of less than 1 micron, less than 100 nm, less than 10 nm, or less than 1 nm. In some cases, one or more regions may be a single monolayer of atoms (i.e., “delta-doping”). In certain cases, the region may be less than a single monolayer thick (for example, if some of the atoms within the monolayer are absent).

The two or more regions may be longitudinally arranged relative to each other, and/or radially arranged (e.g., as in a core/shell arrangement) within the nanoscale wire. As one example, the nanoscale wire may have multiple regions of semiconductor materials arranged longitudinally. In another example, a nanoscale wire may have two regions having different compositions arranged longitudinally, surrounded by a third region or several regions, each having a composition different from that of the other regions. As a specific example, the regions may be arranged in a layered structure within the nanoscale wire, and one or more of the regions may be delta-doped or at least partially delta-doped. As another example, the nanoscale wire may have a series of regions positioned both longitudinally and radially relative to each other. The arrangement can include a core that differs in composition along its length (changes in composition or concentration longitudinally), while the lateral (radial) dimensions of the core do, or do not, change over the portion of the length differing in composition. The shell portions can be adjacent each other (contacting each other, or defining a change in composition or concentration of a unitary shell structure longitudinally), or can be separated from each other by, for example, air, an insulator, a fluid, or an auxiliary, non-nanoscale wire component. The shell portions can be positioned directly on the core, or can be separated from the core by one or more intermediate shells portions that can themselves be constant in composition longitudinally, or varying in composition longitudinally, i.e., the invention allows the provision of any combination of a nanowire core and any number of radially-positioned shells (e.g., concentric shells), where the core and/or any shells can vary in composition and/or concentration longitudinally, any shell sections can be spaced from any other shell sections longitudinally, and different numbers of shells can be provided at different locations longitudinally along the structure.

In some embodiments, a nanoscale wire may be positioned proximate the surface of a substrate, i.e., the nanoscale wire may be positioned within about 50 nm, about 25 nm, about 10 nm, or about 5 nm of the substrate. In some cases, the proximate nanoscale wire may contact at least a portion of the substrate. In one embodiment, the substrate comprises a semiconductor and/or a metal. Non-limiting examples include Si, Ge, GaAs, etc. In certain embodiments, the substrate may comprise a nonmetal and/or nonsemiconductor material, for example, a glass, a plastic or a polymer, a gel, a thin film, etc. Non-limiting examples of suitable polymers that may form or be included in the substrate include polyethylene, polypropylene, poly(ethylene terephthalate), polydimethylsiloxane, or the like. In addition, in some embodiments, one or more contacts for interacting with the nanoscale wire may be defined on the substrate, e.g., using well-established techniques known to those of ordinary skill in the art. For example, one or more regions may be defined on a substrate to act as a source and/or drain for a transistor, e.g., a field-effect transistor.

In some embodiments, the nanoscale wire may be bent or kinked. See, e.g., Int. Pat. Apl. Ser. No. PCT/US10/50199, filed Sep. 24, 2010, entitled “Bent Nanowires and Related Probing of Species,” published as Int. Pat. Apl. Pub. No. WO 2011/038228 on Mar. 31, 2011, incorporated herein by reference in its entirety. In various embodiments, the nanoscale wire may have a shape such that an imaginary straight line connecting two points of the nanoscale wire farthest away from each other along the nanoscale wire may, or may not, exit the nanoscale wire.

Also provided, according to certain aspects of the invention, is a sensing element comprising a nanoscale wire and a detector constructed and arranged to determine a property and/or a change in a property of the nanoscale wire. In some cases, alteration of a property of the nanoscale wire may be indicative of an interaction between a reaction entity and an analyte (e.g., association or dissociation of the reaction entity and the analyte). Where a detector is present, any detector capable of determining a property associated with the nanoscale wire can be used. Examples of electrical or magnetic properties that can be determined include, but are not limited to, voltage, current, conductivity, resistance, impedance, inductance, charge, etc.

Thus, for example, the nanoscale wire may be arranged to form the gate of a field-effect transistor, e.g., between a gate electrode and a drain electrode. For instance, binding of an analyte to the nano scale wire, e.g., as discussed herein, may affect the gating properties of the nanoscale wire, which can be determined as changes in electrical properties of the nanoscale wire within the field-effect transistor.

In one set of embodiments, at least a portion of the nanoscale wire is addressable by a sample (e.g., a gas or liquid sample) containing, or at least suspected of containing, the analyte. The term “addressable,” e.g., by a fluid, is defined as the ability of the fluid to be positioned relative to the nanoscale wire so that the analytes suspected of being in the fluid are able to interact with the nanoscale wire. The fluid may be proximate to or in contact with the nanoscale wire. In some embodiments, the fluid may be directed to the nanoscale wire through the use of a microfluidic channel, e.g., as further described below.

In one embodiment, a conductance (or a change in conductance) less than about 1 nS in a nanoscale wire sensor of the invention can be detected. In another embodiment, a conductance in the range of thousandths of a nS can be detected. In other embodiments, conductances of less than about 10 microsiemens, less than about 1 microsiemen, less than about 100 nS, or less than about 10 nS can be detected. The concentration of a species, or analyte, may be detected from femtomolar concentrations, to nanomolar, micromolar, millimolar, and to molar concentrations and above.

As a non-limiting example, a charged analyte may be determined by determining a change in an electrical property of the nanoscale wire, for example, conductivity. Immobilizing a charged analyte relative to the nanoscale wire may cause a change in the conductivity of the nanoscale wire, and in some cases, the distance between the charged analyte and the nanoscale wire may determine the magnitude of the change in conductivity of the nanoscale wire. Uncharged analytes can be similarly determined, for instance, by causing the analyte to become charged, e.g., by altering environmental conditions such as pH (by raising or lowering pH), temperature, reactants, or the like, by reacting the analyte with a charged moiety, or the like.

The analyte to be determined by the nanoscale sensor may be present within a sample. The term “sample” refers to any cell, lysate, tissue, or fluid from a biological source (a “biological sample”), or any other medium, biological or non-biological, that can be evaluated in accordance with the invention. The sample may be, for instance, a liquid (e.g., a solution or a suspension) or a gas. A sample includes, but is not limited to, a biological sample drawn from an organism (e.g. a human, a non-human mammal, an invertebrate, a plant, a fungus, an algae, a bacteria, a virus, etc.), a sample drawn from food designed for human consumption, a sample including food designed for animal consumption such as livestock feed, milk, an organ donation sample, a sample of blood destined for a blood supply, a sample from a water supply, a soil sample, or the like.

In some cases, the sample may be a sample suspected of containing an analyte. A “sample suspected of containing” a particular component means a sample with respect to which the content of the component is unknown. For example, a fluid sample from a human suspected of having a disease, but not known to have the disease, defines a sample suspected of containing the disease. “Sample” in this context includes naturally-occurring samples, such as physiological samples from humans or other animals, samples from food, livestock feed, water, soil, etc. Typical samples include tissue biopsies, cells, cell lysates, whole blood, serum or other blood fractions, urine, ocular fluid, saliva, fluid or other samples from tonsils, lymph nodes, needle biopsies, etc.

A variety of sample sizes, for exposure of a sample to a nanoscale sensor of the invention, can be used in various embodiments. As examples, the sample size used in nanoscale sensors may be less than or equal to about 10 microliters, less than or equal to about 1 microliter, or less than or equal to about 0.1 microliter. The sample size may be as small as about 10 nanoliters, 1 nanoliter, or less, in certain instances. The nanoscale sensor also allows for unique accessibility to biological species and may be used for in vivo and/or in vitro applications. When used in vivo, in some case, the nanoscale sensor and corresponding method result in a minimally invasive procedure.

The invention, in some embodiments, involves a sensing element comprising a sample exposure region and a nanoscale wire able to detect the presence or absence of an analyte, and/or the concentration of the analyte. The “sample exposure region” may be any region in close proximity to the nanoscale wire where a sample in the sample exposure region addresses at least a portion of the nanoscale wire. Examples of sample exposure regions include, but are not limited to, a well, a channel, a microfluidic channel, or a gel. In certain embodiments, the sample exposure region is able to hold a sample proximate the nanoscale wire, and/or may direct a sample toward the nanoscale wire for determination of an analyte in the sample. The nanoscale wire may be positioned adjacent or within the sample exposure region. Alternatively, the nanoscale wire may be a probe that is inserted into a fluid or fluid flow path. Fluid flow channels can be created at a size and scale advantageous for use in the invention (microchannels) using a variety of techniques such as those described in International Patent Application Serial No. PCT/US97/04005, entitled “Method of Forming Articles and Patterning Surfaces via Capillary Micromolding,” filed Mar. 14, 1997, published as Publication No. WO 97/33737 on Sep. 18, 1997, and incorporated herein by reference.

As an example, a sample, such as a fluid suspected of containing an analyte that is to be determined, may be presented to a sample exposure region of a sensing element comprising a nanoscale wire. An analyte present in the fluid that is able to bind to the nanoscale wire and/or a reaction entity immobilized relative to the nanoscale wire may cause a change in a property of the nanoscale wire that is determinable upon binding, e.g. using conventional electronics. If the analyte is not present in the fluid, the relevant property of the nanoscale wire will remain unchanged, and the detector will measure no significant change. Thus, according to this particular example, the presence or absence of an analyte can be determined by monitoring changes, or lack thereof, in the property of the nanoscale wire. In some cases, if the detector measures a change, the magnitude of the change may be a function of the concentration of the analyte, and/or a function of some other relevant property of the analyte (e.g., charge or size, etc.). Thus, by determining the change in the property of the nanoscale wire, the concentration or other property of the analyte in the sample may be determined.

In some embodiments, one or more nanoscale wires may be positioned in a channel or in a microfluidic channel, which may define the sample exposure region in some cases. As used herein, a “channel” is a conduit that is able to transport one or more fluids specifically from one location to another. Materials may flow through the channels, continuously, randomly, intermittently, etc. The channel may be a closed channel, or a channel that is open, for example, open to the external environment. The channel can include characteristics that facilitate control over fluid transport, e.g., structural characteristics, physical/chemical characteristics (e.g., hydrophobicity vs. hydrophilicity) and/or other characteristics that can exert a force (e.g., a containing force) on a fluid when within the channel. The fluid within the channel may partially or completely fill the channel. In some cases the fluid may be held or confined within the channel or a portion of the channel in some fashion, for example, using surface tension (i.e., such that the fluid is held within the channel within a meniscus, such as a concave or convex meniscus). The channel may have any suitable cross-sectional shape that allows for fluid transport, for example, a square channel, a circular channel, a rounded channel, a rectangular channel (e.g., having any aspect ratio), a triangular channel, an irregular channel, etc. The channel may be of any size. For example, the channel may have a largest dimension perpendicular to a direction of fluid flow within the channel of less than about 1000 micrometers in some cases (i.e., a microfluidic channel), less than about 500 micrometers in other cases, less than about 400 micrometers in other cases, less than about 300 micrometers in other cases, less than about 200 micrometers in still other cases, less than about 100 micrometers in still other cases, or less than about 50 or 25 micrometers in still other cases. In some embodiments, the dimensions of the channel may be chosen such that fluid is able to freely flow through the channel. The dimensions of the channel may also be chosen in certain cases, for example, to allow a certain volumetric or linear flowrate of fluid within the channel. Of course, the number of channels, the shape or geometry of the channels, and the placement of channels can be determined by those of ordinary skill in the art.

One or more different nanoscale wires may cross the same microfluidic channel (e.g., at different positions) to detect the same or different analytes, to measure a flowrate of an analyte(s), etc. In another embodiment, one or more nanoscale wires may be positioned in a microfluidic channel to form one of a plurality of analytic elements, for instance, in a microneedle probe, a dip and read probe, etc. The analytic elements probe may be implantable and capable of detecting several analytes simultaneously in real time, according to certain embodiments. In another embodiment, one or more nanoscale wires may be positioned in a microfluidic channel to form an analytic element in a microarray for a cassette or a lab-on-a-chip device. Those of ordinary skill in the art would know of examples of cassette or lab-on-a-chip devices that are suitable for high-throughput chemical analysis and screening, combinational drug discovery, etc. The ability to include multiple nanoscale wires in one nanoscale sensor also allows, in some cases, for the simultaneous detection of different analytes suspected of being present in a single sample, i.e., the nanoscale sensor allows “multiplexed” detection of different analytes. For example, a nanoscale sensor may include a plurality of nanoscale wires that each detects different pH levels, proteins, enzymes, toxins, small molecules, and/or nucleic acids, etc.

In some cases, the sensing element may comprise a plurality of nanoscale wires able to determine (i.e., detect the presence, absence, and/or amount or concentration) one or more analytes within a sample, for example, from a liquid or solution, blood serum, etc., as previously described. Various nanoscale wires within the sensing element may be differentially doped as described herein, and/or contain different reaction entities, and/or the same reaction entities at different concentrations, thereby varying the sensitivity of the nanoscale wires to the analytes, as needed. For example, different reaction entities may be “printed” on the nanoscale wires, e.g., using microarray printing techniques or the like, thereby producing an array of nanoscale wires comprising different reaction entities. In some cases, individual nanoscale wires may be selected based on their ability to interact with specific analytes, thereby allowing the detection of a variety of analytes. The plurality of nanoscale wires may be randomly oriented or parallel to one another, according to another set of embodiments. The plurality of nanoscale wires may also be oriented in an array on a substrate, in specific instances.

A sensing element of the present invention can collect real time data and/or near-real time data, in some embodiments. The data may be used, for example, to monitor the reaction rate of a specific chemical or biological reaction. Physiological conditions or drug concentrations present in vivo may also produce a real time (or near-real time) signal that may be used to control a drug delivery system, in another embodiment of the invention. In addition, electrical determination of one or more properties of the nanoscale wire may allow for the determination of one or more analytes as a function of time. For example, the conductance of a nanoscale wire may be determined as a function of time, which may give additional information regarding the analyte.

In some cases, the nanoscale wires, or at least a portion of the nanoscale wires, may be individually addressable, i.e., the status of the nanoscale wire may be determined without determining the status of nearby nanoscale wires. Thus, for example, a nanoscale wire within a sensing element, or a number of nanoscale wires within the sensing element, may be in electrical communication with an electrode that is able to address the nanoscale wire(s), and such a wire may be addressed using the electrode without addressing other nanoscale wires not in electrical communication with the electrode. For example, a first reaction entity immobilized relative to a first nano scale wire may bind an analyte, and such a binding event may be detectable independently of the detection of a binding event involving a second reaction entity immobilized relative to a second nanoscale wire. The electrodes may be in electronic communication with one or more electrical contacts.

In some embodiments, the invention includes a microarray including a plurality of sensing regions, at least some of which comprise one or more nanoscale wires. The microarray, including some or all of the sensing regions, may define a sensing element in a sensor device. At least some of the nanoscale wires are able to determine an analyte suspected to be present in a sample that the sensing region of the microarray is exposed to, for example, the nanoscale wire may comprise a reaction entity able to interact with an analyte. If more than one nanoscale wire is present within the sensing region, the nanoscale wires may be able to detect the same analyte and/or different analytes, depending on the application.

In addition, in certain cases, the nanoscale wire may be part of a larger structure, such as an array or a scaffold. Examples of scaffolds comprising nanoscale wires include those discussed in U.S. Pat. Apl. Pub. Nos. 2014/0073063 or 2014/0074253, or Int. Pat. Apl. Pub. No. WO 2014/165634, each incorporated herein by reference in its entirety. For example, one set of embodiments is generally directed to a scaffold, such as a tissue scaffold, containing nanoscale wires such as those discussed herein.

Various embodiments of the present invention find use in a wide range of applications. For instance, in some aspects, any of the techniques described herein may be used in the determination of proteins, enzymes, toxins, viruses, small molecules, or the like, e.g., as in an assay, for example, to detect or diagnose cancer or other medical conditions, toxins or other environmental agents, viruses, or the like. A property of an analyte may be determined by allowing the analyte to interact with a nanoscale wire and/or a reaction entity, and the interaction may be analyzed or determined in some fashion, e.g., quantified. In some cases, the degree or amount of interaction (e.g., a binding constant) may be determined, for example, by measuring a property of the nanoscale wire (e.g., an electronic property, such as the conductance) after exposing the nanoscale wire and/or the reaction entity to the analyte.

In certain instances, such assays are useful in drug screening techniques. In one example, a protein, enzyme, or other target molecule may be immobilized relative to a nanoscale wire as a reaction entity, and exposed to one or more drug candidates, for example, serially or simultaneously. Interaction of the drug candidate(s) with the reaction entity may be determined by determining a property of the nanoscale wire, e.g., as previously described. As a non-limiting example, a nanoscale wire, having an associated reaction entity, may be exposed to one or more species able to interact with the reaction entity, for instance, the nanoscale wire may be exposed to a sample containing a first species able to interact with the reaction entity, where the sample contains or is suspected of containing a second species able to interact with the reaction entity, and optionally other, different species, where one of the species is a drug candidate. As one example, if the reaction entity is an enzyme, the sample may contain a substrate and a drug candidate suspected of interacting with the enzyme in a way that inhibits enzyme/substrate interaction; if the reaction entity is a substrate, the sample may contain an enzyme and a drug candidate suspected of interacting with the substrate in an inhibitory manner; if the reaction entity is a nucleic acid, the sample may contain an enzyme able to bind the nucleic acid (e.g., a nucleic acid synthesis enzyme), or a complementary nucleic acid, and a drug candidate suspected of interacting with the nucleic acid reaction entity in an inhibitory manner; if the reaction entity is a receptor, the sample may contain a ligand for the receptor and a drug candidate suspected of interacting with the receptor in an inhibitory manner; etc. In each of these cases, the drug candidate may also act in a way that enhances, rather than inhibits, interaction.

In some cases, assays of the invention may be used in high-throughput screening applications, e.g., where at least 100, at least 1,000, at least 10,000, or at least 100,000 or more analytes may be rapidly screened, for example, by exposing one or more analytes to a nanoscale wire (e.g., in solution), and/or exposing a plurality of analytes to a plurality of nanoscale wires and/or reaction entities.

In one aspect, the present invention provides any of the above-mentioned devices packaged in kits, optionally including instructions for use of the devices. As used herein, “instructions” can define a component of instructional utility (e.g., directions, guides, warnings, labels, notes, FAQs (“frequently asked questions”), etc., and typically involve written instructions on or associated with packaging of the invention. Instructions can also include instructional communications in any form (e.g., oral, electronic, digital, optical, visual, etc.), provided in any manner such that a user will clearly recognize that the instructions are to be associated with the device, e.g., as discussed herein. Additionally, the kit may include other components depending on the specific application, for example, containers, adapters, syringes, needles, replacement parts, etc. As used herein, “promoted” includes all methods of doing business including, but not limited to, methods of selling, advertising, assigning, licensing, contracting, instructing, educating, researching, importing, exporting, negotiating, financing, loaning, trading, vending, reselling, distributing, replacing, or the like that can be associated with the methods and compositions of the invention, e.g., as discussed herein. Promoting may also include, in some cases, seeking approval from a government agency to sell a composition of the invention for medicinal purposes. Methods of promotion can be performed by any party including, but not limited to, businesses (public or private), contractual or sub-contractual agencies, educational institutions such as colleges and universities, research institutions, hospitals or other clinical institutions, governmental agencies, etc. Promotional activities may include instructions or communications of any form (e.g., written, oral, and/or electronic communications, such as, but not limited to, e-mail, telephonic, facsimile, Internet, Web-based, etc.) that are clearly associated with various embodiments of the invention.

The following documents are each incorporated herein by reference in their entireties: U.S. patent application Ser. No. 10/020,004, entitled “Nanosensors,” by Lieber, et al., now U.S. Pat. No. 7,129,554, issued Oct. 31, 2006; U.S. patent application Ser. No. 11/082,372, entitled “Doped elongated semiconductors, growing such semiconductors, devices including such semiconductors and fabricating such devices,” by Lieber, et al., now U.S. Pat. No. 7,211,464, issued May 1, 2007; U.S. patent application Ser. No. 10/196,337, entitled “Nanoscale wires and related devices,” by Lieber, et al., now U.S. Pat. No. 7,301,199, issued Nov. 27, 2007; U.S. patent application Ser. No. 12/536,269, entitled “Nanoscale sensors,” by Lieber, et al., now U.S. Pat. No. 8,232,584, issued Jul. 31, 2012; U.S. patent application Ser. No. 12/312,740, entitled “High-sensitivity nanoscale wire sensors,” by Lieber, et al., now U.S. Pat. No. 8,575,663, issued Nov. 5, 2013; U.S. patent application Ser. No. 12/225,142, entitled “Nanobioelectronics,” by Patolsky, et al., published as U.S. Pat. Apl. Pub. No. 2009/0299213 on Dec. 3, 2009; U.S. patent application Ser. No. 12/308,207, entitled “Nanosensors and related technologies,” by Lieber, et al., published as U.S. Pat. Apl. Pub. No. 2010/0087013 on Apr. 8, 2010; U.S. patent application Ser. No. 13/497,852, entitled “Bent nanowires and related probing of species,” by Tian, et al., published as U.S. Pat. Apl. Pub. No. 2012/0267604 on Oct. 25, 2012; U.S. patent application Ser. No. 14/018,075, entitled “Methods and systems for scaffolds comprising nanoelectronic components,” by Lieber, et al., published as U.S. Pat. Apl. Pub. No. 2014/0073063 on Mar. 13, 2014; U.S. patent application Ser. No. 14/018,082, entitled “Scaffolds comprising nanoelectronic components for cells, tissues, and other applications,” by Lieber, et al., published as U.S. Pat. Apl. Pub. No. 2014/0074253 on Mar. 13, 2014; U.S. patent application Ser. No. 14/124,816, entitled “Nanoscale wires, nanoscale wire FET devices, and nanotube-electronic hybrid devices for sensing and other applications,” by Lieber, et al., published as U.S. Pat. Apl. Pub. No. 2014/0184196 on Jul. 3, 2014; Int. Pat. Apl. Ser. No. PCT/US2013/039228, entitled “Nanoscale sensors for intracellular and other applications,” by Lieber, et al., published as Int. Pat. Apl. Pub. No. WO 2013/166259 on Nov. 7, 2013; and Int. Pat. Apl. Ser. No. PCT/US2013/059454, entitled “Nanoscale field-effect transistors for biomolecular sensors and other applications,” by Lieber, et al., published as Int. Pat. Apl. Pub. No. WO 2014/043341 on Mar. 20, 2014.

The following examples are intended to illustrate certain embodiments of the present invention, but do not exemplify the full scope of the invention.

### EXAMPLE 1

Transistor-based nanoelectronic sensors are capable of label-free, real-time chemical and biological detection with high sensitivity and spatial resolution, although the short Debye screening length in high ionic strength solutions has made difficult applications relevant to physiological conditions. This example describes a general strategy for field-effect transistor (FET) sensors that involves incorporating a porous and biomolecule permeable polymer layer on the FET sensor. This polymer layer increases the effective screening length in the region immediately adjacent to the device surface, and thereby allows detection of biomolecules in high ionic strength solutions, e.g., in real-time. These studies of silicon nanowire FETs with and without additional polyethylene glycol (PEG) modification show that prostate specific antigen (PSA) can be readily detected in solutions with phosphate buffer (PB) concentrations as high as 150 mM, while similar devices without PEG modification only exhibit detectable signals for concentrations of less than about 10 mM.

Concentration-dependent measurements exhibited real-time detection of PSA to at least 10 nM in ˜130 mM ionic strength PB with a linear response up to the highest (1000 nM) PSA concentrations tested. This example represents an important step toward general application of nanoelectronic detectors for biochemical sensing in physiological environments, including in vitro and in vivo biological sensing.

Since the first demonstration of biosensors based on nanoelectronic field-effect transistors (FETs), Si nanowire (SiNW) FET biosensors have been used for ultrasensitive, multiplexed real-time detection of a variety of biological species including protein disease biomarkers, DNA and DNA mismatch identification, and single viruses. However, these sensors also have been limited to measurements in relatively low ionic strength solutions by the Debye-screening length. Specifically, in physiological solution environments, which are relevant to many important biological, medical, and diagnostic applications, the short screening length, less than 1 nm, may reduce the field produced by charged biomolecules at FET surface and thus may make difficult real-time label-free detection.

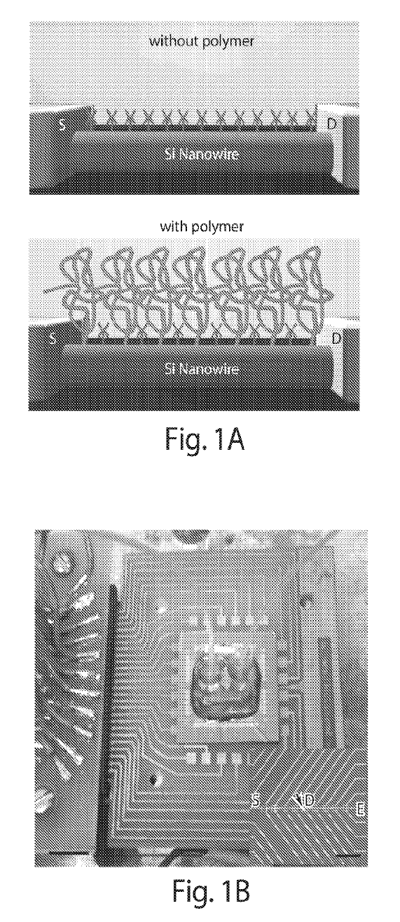

These examples illustrate a new strategy for real-time detection of biomolecules in physiological environment using nanoscale FET device and applicable to both in vitro and in vivo sensing. FIG. 1A involves linking a porous and biomolecule permeable polymer to the surface of the FET such that the effective Debye screening length is increased, making it possible to detect proteins and other biological analytes directly in real-time in physiology-relevant high ionic strength solutions. In these examples, this is demonstrated for SiNW FET devices modified with PEG, although it should be noted that this approach will also work for FET and other biosensors configured using other materials as well.

SiNWs (p-type, 30 nm diameter) synthesized by nanocluster catalyzed vapor-liquid-solid methods were used to fabricate FET sensor chip (FIG. 1B). See, e.g., U.S. Pat. Nos. 7,211,464; 7,129,554; 7,301,199; 8,232,584, each incorporated herein by reference in its entirety. Arrays of Si-NW devices were defined using photolithography with Ti/Pd/Ti (1.5 nm/55 nm/7 nm) contacts deposited by thermal evaporation on silicon substrates with a 600 nm-thick oxide layer. The metal contacts and interconnects were passivated with a ˜60 nm thick Si₃N₄ layer deposited by magnetron sputtering.

The SiNW source-drain contacts and metal interconnects were passivated with Si₃N₄. The SiNW device chips were modified with either a 4:1 mixture of (3-aminopropyl)triethoxysilane (APTES) and silane-PEG (10 kD) or pure APTE. The surface modification process for 4:1 APTES:silane-PEG and pure APTES (FIG. 5) was carried out as follows. APTES (741442, Sigma-Aldrich, St Louis, Mo.) and silane-PEG (methoxyl silane PEG, No. PG1-SL-10k, Nanocs, Inc., Boston, Mass.) were first dissolved in the mixture of 95% ethanol and 5% DI water with a final concentration of APTES and silane-PEG of 4.8 mM and 1.2 mM, respectively; pure APTES solutions were made in a similar manner with only APTES. Precleaned SiNW device chips were reacted with the ethanol solutions of APTES/silane-PEG or APTES for 45 minutes, and then washed gently with ethanol and allowed to dry.

FIG. 5 shows a schematic of a nanowire surface modification with polymer. The top portion shows an illustration of the silicon nanowire SiO₂ surface with hydroxyl group termination. The bottom portion shows a schematic of the co-modification strategy using a mixture of APTES and silane-PEG (triethoxysilane-PEG; MW=10 kD). An APTES:silane-PEG (4:1) mixture was used in these studies. Immediately prior to modification, the silicon nanowire device chip was cleaned with oxygen plasma (40 W for 60 sec), and then the chip was reacted with a silane/ethanol (95:5, EtOH:H₂O) solution to yield the modified nanowire surface.

Following modification of the SiNW device chip, a poly(dimethylsiloxane) (PDMS) microfluidic channel was mounted on the chip (FIG. 1B) for delivery of buffer and protein/buffer solutions. The SiNW devices were initially characterized using a probe station, and then wire-bonded to the output pads of the chip carrier. A PDMS microfluidic channel was mounted on the sensor chip, with the channel aligned and overlapped to the central region of the device chip where the SiNW FETs are located. Polyethylene tubing was attached to the inlet and the outlet holes on the PDMS microfluidic channel, and PB or PSA/PB solution was drawn through the channel using a syringe pump at 0.2 ml/h. Measurements were carried out using up to 3 independent lock-in amplifiers (modulation frequencies of 79, 97, and 103 Hz) to record 3 different SiNW FET elements selected from 188 elements on the chip. The conductance versus time data were digitized and recorded on computer using custom software. The water gate responses of devices were characterized before PSA detection experiments. Device transconductance values were determined from water gate measurements, and used to convert recorded conductance-time data to mV values.

The SiNW FET signals were recorded simultaneously from three devices, and signals were converted to absolute millivolt (mV) values using the device transconductance sensitivities determined from water gate measurements. Prostate specific antigen (PSA) was used as a protein model with all experiments carried out below the PSA isoelectric point at pH 6 PB. Preservative free PSA (MBS537240, Mybiosource, Inc., San Diego, Calif.) was used without further purification and directly diluted in different ionic strength pH 6 PB prior to sensing measurements.

FIG. 1 shows polymer surface modification to increase the effective Debye length for FET biosensing. FIG. 1A is a schematic illustration of a NW FET device (top) without and (bottom) with a porous and biomolecule permeable polymer surface modification. The features on both NW surfaces represent APTES in these studies, or more generally, specific receptors. FIG. 1B is an optical image of device chip (central light square, ca. 2×2 cm²) mounted on a PCB interface board that was plugged into the input/output interface connected (left side of image) to a computer controlled data acquisition system (not shown). The copper squares surrounding the device chip were connected to the chip by wire-bonding (not visible). A PDMS-based microfluidic channel was mounted over the central SiNW device region of the chip with solution input/output via tubing (translucent, center to upper left/right of image) during real-time biosensing experiments. The inset shows a bright-field microscopy image of a portion of device chip containing 18 of 188 total FET devices on the chip; scale bar is 40 micrometers. Metal lines are visible in the image with common source (S) and one addressable drain (D) electrode labeled; the other addressable D electrodes are visible as the thin gold lines oriented upwards and downwards to right. The arrow in the center of the inset highlights one SiNW FET as shown schematically in FIG. 1A.

### EXAMPLE 2

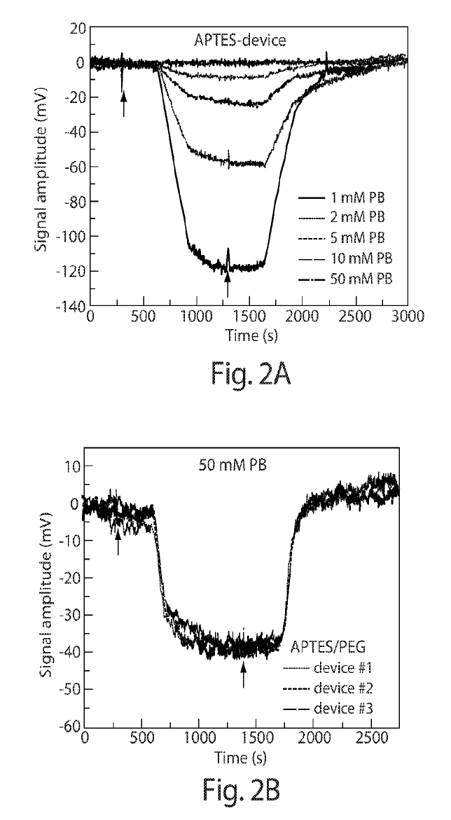

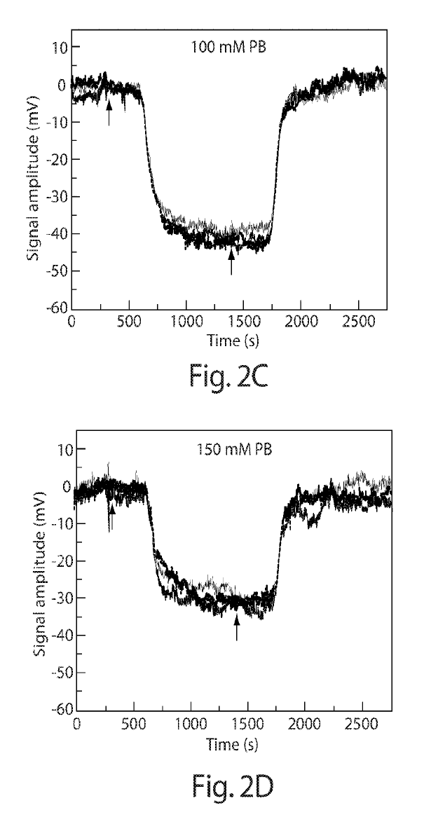

In this example, initial measurements made as a function PB concentration on APTES modified SiNW devices (FIG. 2A) show that the PSA signal response is significantly diminished with increasing PB buffer ionic strength. First, as the PB concentration was increased from 1, 2, 5, to 10 mM, the signal response for 100 nM PSA dramatically decreased from ca. 112, 56, 23 to 8 mV, respectively. Second, when the PB concentration was further increased to 50 mM, no obvious response was observed. These results generally showed a decrease in FET signal amplitudes with increasing solution ionic strength, and indicated that the APTES modification has little influence on the local ionic environment at the FET surface.

FIG. 2 shows real-time PSA detection using SiNW FETs sensors with and without PEG surface modifications. FIG. 2A shows signal amplitude vs time data recorded from an APTES modified SiNW FET following addition of 100 nM PSA (initial arrow) and pure buffer (following arrow) for different pH 6 PB concentrations. FIGS. 2B to 2D show comparison of signal response traces recorded simultaneously from three SiNW FET devices following addition of 100 nM PSA in different concentration pH 6 PBs; the devices were modified with 4:1 APTES/silane-PEG. The PB concentrations of the pure and PSA/PB solutions in FIGS. 2B, 2C, and 2D were 50, 100, and 150 mM, respectively. The arrows in figure correspond to the points where the solution flow was switched from pure buffers to protein solutions and from protein solutions to pure buffers; the delay from solution switch to signal change corresponds to the time for solution to flow from the entry point to the devices in this set-up.

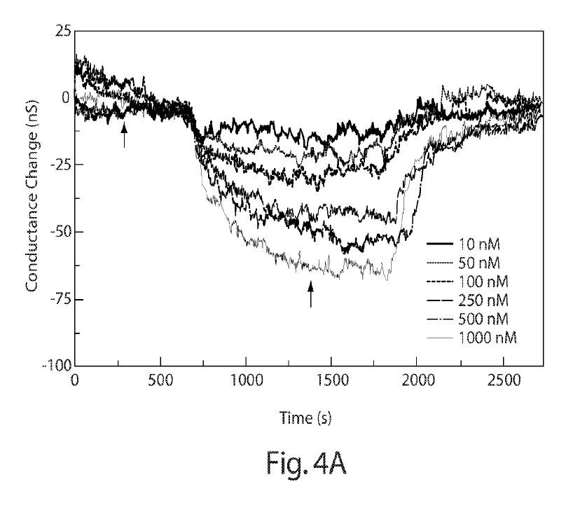

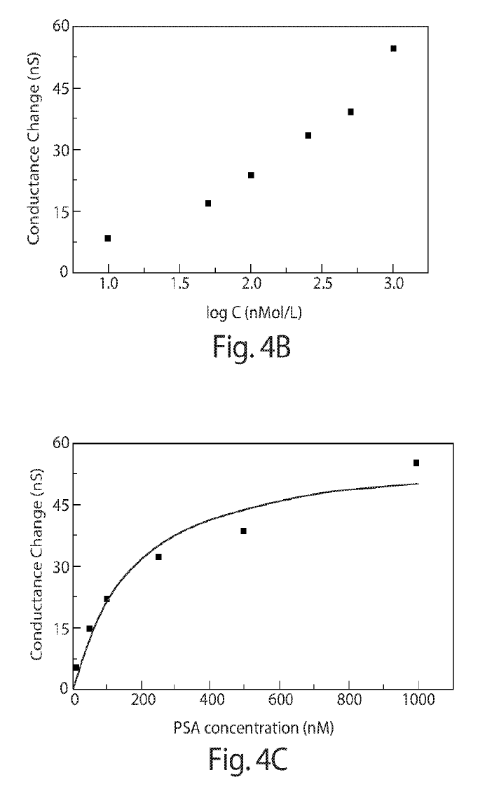

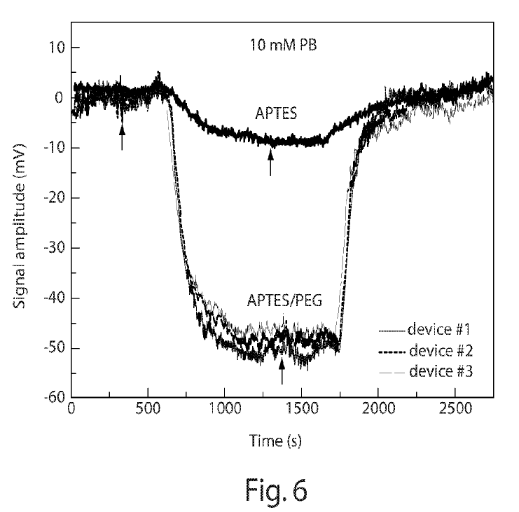

Measurements made with polymer-modified SiNW FET devices (FIGS. 4A-4D and 6) exhibited well-defined PSA signal responses for PB concentrations up to 150 mM and highlight several points. First, measurements showed a weak PB concentration dependence with response of ca. 44, 37, 40, and 28 mV for 10, 50, 100, and 150 mM PB, respectively. It was noted that no obvious signals were detected for >10 mM PB under same experimental conditions using only APTES-modified devices. Second, the data recorded for each PB concentration was reproducible, as evidenced by similar responses recorded simultaneously from three different modified SiNW devices on the sensor chip. Third, the ionic strengths of the higher concentration solutions were similar to physiological conditions, and thus showed the potential for direct real-time sensing in this regime.

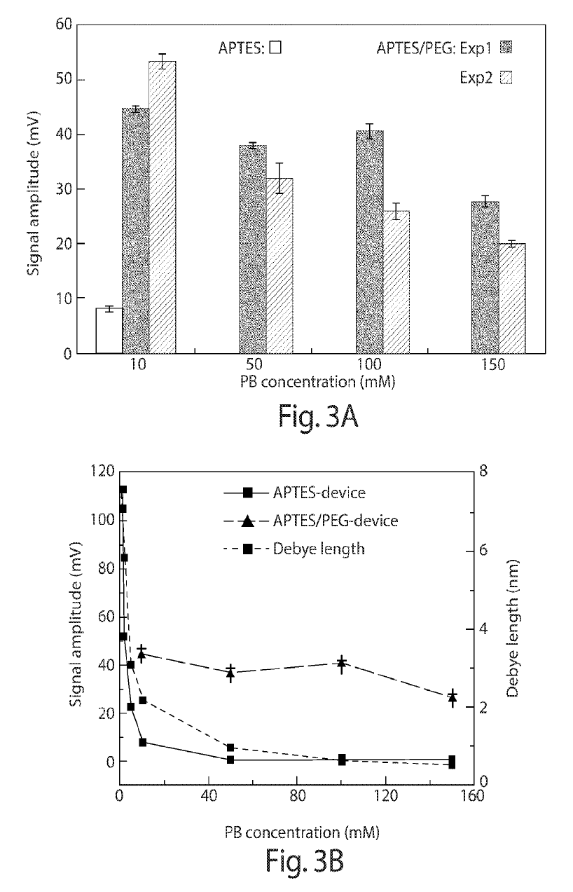

The reproducibility of PSA sensing at different PB concentrations was investigated further with polymer-modified sensors by comparing the results obtained from independent SiNW sensor chips. Notably, the signals recorded from three independent devices on each of two different sensor chips (FIG. 3A) exhibited good consistency at all PB concentrations (i.e., 10, 50, 100, and 150 mM). These results indicated that the APTES/silane-PEG surface modification was relatively uniform, although there is a ca. 14% variation of the PSA signal between the two different sensor chips. It is believed that this chip-to-chip variation, which may reflect variations in the PEG density/porosity obtain using this modification approach, can be reduced, e.g., through optimization of the post-fabrication cleaning and modification strategies.

Comparison of these PB concentration-dependent experimental sensing data and the calculated Debye length as a function of PB concentration (FIG. 3B) brings out several key points. For APTES-modified devices, the PSA (100 nM) response in 1 mM PB with a Debye length of ˜7 nm was 112 mV, and decreased rapidly to ca. 8 mV in 10 mM PB where the Debye length was 2.2 nm. Moreover, no obvious response was observed in 50 mM PB where the Debye length was ˜1 nm. This rapid decrease and ultimate loss of signal response is consistent with the thickness of the APTES monolayer, 0.8 nm, and the diameter of PSA, ca. 2 nm, since charge screening at the FET surface will become increasingly effective as the Debye length becomes smaller than the sum of the APTES layer thickness and protein diameter. In contrast, for the APTES/PEG-modified devices, the PSA (100 nM) response in 10 mM PB was 44 mV (vs. 8 mV for APTES alone), and moreover, decreased only slowly to ca. 40 mV for 100 mM PB (Debye length ˜0.67 nm) and 28 mV for 150 mM PB where the Debye length is ˜0.54 nm. The difference between the APTES and APTES/PEG modified devices revealed that the polymer plays an important role in modulating the local ionic environment at the SiNW FET surfaces, and thus overcoming Debye screening for FET biosensors under physiologically-relevant conditions.

FIG. 3 shows reproducible PSA detection by FET sensors in high ionic strength solutions. FIG. 3A shows a comparison of PSA response signals from independent sensor chips as labeled in the figure, where APTES/PEG corresponds to modification with 4:1 APTES/silane-PEG. The error bars for each experiment correspond to +/−1 standard deviation from data acquired simultaneously from three independent devices. FIG. 3B shows the dependence of PSA signal amplitudes and Debye length on the PB concentrations. The SiNW FET functionalization is the same as in FIG. 3A.

FIG. 6 shows a comparison of PSA detection in 10 mM PB for devices with and without polymer modification. The time-dependent nanowire device responses are shown for nanowires modified with a 4:1 mixture of APTES:silane-PEG (devices #1 to #3) or pure APTES (black trace). The data for the three polymer modified devices were recorded simultaneously from the same chip, while that for the pure APTES device was recorded in a separate experiment. The PSA concentration in all experiments was 100 nM; the first arrow (left) indicates the time that the PSA/10 mM PB solution was added to solution delivery line, and the other arrows (center of image) shows the point at which the solution was changed to pure 10 mM PB.

### EXAMPLE 3

In this example, the PSA concentration dependent sensor response was investigated under high ionic strength physiological conditions. Conductance versus time data recorded in 100 mM PB (FIG. 4A), which has a Debye length ˜0.67 nm comparable with physiological solution, showed good sensor response for PSA concentrations from 10-1000 nM. A standard plot of this data versus log[PSA] (FIG. 4B) yielded a relatively linear detection regime for PSA concentrations greater than or equal to 50 nM.

The PSA concentration-dependent sensor response in terms of Langmuir Adsorption was also investigated. It was found that the data can be well fit by the expression for a Langmuir adsorption isotherm (equation 1):

$$S = {S}_{max} \frac{k C}{1 + k C} \qquad ( 1 )$$

where S and Sₘₐₓ represent the signal and saturation signal, respectively, in response to PSA concentration C, and k is a constant. Significantly, the value of k that was determined, 6.4×10⁶ M⁻¹, agrees well with reported results for protein adsorption.

FIG. 4 shows concentration-depended PSA detection in high ionic strength solutions. FIG. 4A shows a time-dependent signal response traces at different PSA concentrations for a PEG-modified SiNW FET sensor in pH 6 100 mM PB. FIG. 4B shows sensor response (conductance change) versus logarithm PSA concentration. FIG. 4C shows a plot of the sensor response vs PSA concentration. The line is a fit of the data with Langmuir adsorption isotherm with k=6.4×10⁶ M⁻¹.

Thus, these examples describe a general strategy to overcome the limitations of Debye screening for FET biomolecule sensors that involves functionalizing the FET with a porous and biomolecule permeable polymer thereby increasing the effective screening length in the region immediately adjacent to the device surface and enabling detection of biomolecules in high ionic strength solutions in real-time. Measurements made with SiNW FETs with and without additional PEG modification show that PSA can be readily detected in solutions with PB concentrations as high as 150 mM, while similar devices without PEG modification only exhibit detectable signals for concentrations of less than or equal to 10 mM. Concentration-dependent measurements exhibited real-time detection of PSA to at least 10 nM in ˜130 mM ionic strength PB with linear response up to the highest (1000 nM) PSA concentrations tested. This could also be applied to other nanowires, carbon nanotube, graphene and 2D semiconductors, or the like. This can also be used for the general application of nanoelectronic detectors in many areas, including real-time point-of-care diagnostics, intracellular and subcellular real-time monitoring of protein and nucleic acids using 3D kinked SiNW FET devices, or in vitro or in vivo monitoring of important biomolecules in engineered and natural tissues using 3D free-standing nanoelectronic scaffolds or networks.

### EXAMPLE 4

In this example, nanoelectronic sensors based on a monolayer graphene field effect transistor were demonstrated to show the similar sensing performance in high ionic strength solutions, with respect to overcoming Debye screening.

Graphene transistors were first fabricated on Si wafer with 600 nm SiO₂ oxide layer, through a serial of standard photolithography steps, which define the graphene channels' length and width to be 10 and 5 micrometers, respectively. 10 nm Cr/65 nm Au electrodes were further deposited as sources and drains, followed by a ca. 40 nm Si₃N₄ deposition onto electrodes as passivation to prevent any potential leaking current between gate electrode (Ag/AgCl reference electrode) and source/drain. Prior to detection of prostate specific antigen (PSA), an altered surface modification strategy was adopted to modify polyethylene glycol (PEG) and ethanolamine onto graphene channels, which is described below.

Graphene sensors were immersed into 0.6 millimolar (mM) pyrenebutyric acid/DMF solution for 1 hour. Graphene sensors were then cleaned with blank DMF at 60° C. on a shaker for 1 hour, to get rid of possible pyrenebutyric acid aggregates on the graphene channel. For modification, a 2 mL mixture of PEG-amino (10 kDa) and ethanolamine, with a total concentration of 3 mM, was prepared in 1× PBS or 10 mM PB, followed by adding 0.8 mg 1-Ethyl-3-(3-dimethylaminopropyl)-carbodiimide (EDC) and 2.2 mg N-hydroxysulfosuccinimide (NHS) as coupling reagents. Note that the ratios between PEG and ethanolamine can be easily controlled to achieve tunable pore sizes in the modified polymer layer.

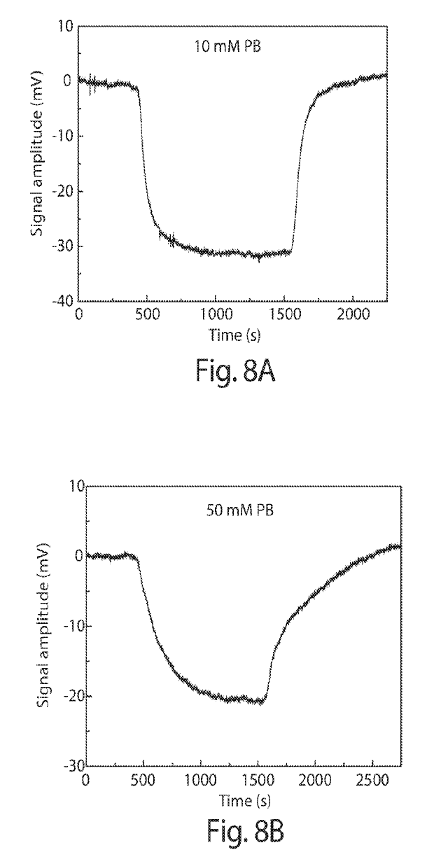

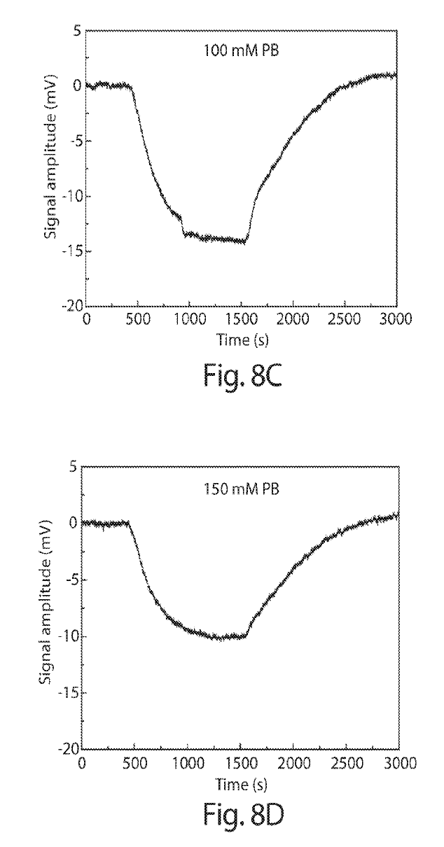

FIG. 8 shows the real-time detection of PSA in 10 mM, 50 mM, 100 mM and 150 mM PB using graphene sensors modified with 1:4 PEG/ethanolamine. As described in silicon nanowire section, 100 nM PSA in 10 mM, 50 mM, 100 mM and 150 mM PB was injected into PDMS microfluidic channels using a syringe pump at 300 s. FIG. 8 illustrates that PSA show good signal-to-noise responses in all PB solutions, which are ca. 30 mV, 20 mV, 14 mV and 10 mV, respectively. In addition, sensing traces showed in a total reversible manner after switching back to PB at 1300 s, similar with those on silicon nanowire sensors.

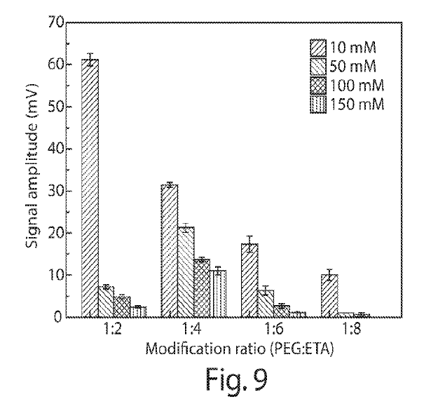

As previously mentioned, the ratio of PEG to ethanolamine could be utilized to control pore size in modified polymer layer, to further facilitate other analytes' detection or optimize modification protocol for a specific analyte, such as PSA. FIG. 9 shows a PEG/ethanolamine ratio dependent PSA signals in high ionic strength PB solutions. From the histogram, graphene sensors with 1:4 PEG/ethanolamine modification presented highest PSA responses in PB, with concentration higher than 50 mM. Graphene sensors modified with PEG/ethanolamine ratio either higher or lower than 1:4 were confirmed to have a decreasing PSA sensing performance. However, in PB with a relatively lower concentration (10 mM), where Debye screening is less effective, PSA shows an incremental signal with PEG/ethanolamine ratio increased from 1:8 to 1:2.
In this example, graphene sensors using a modification strategy similar to that for silicon nanowire sensors also realize good PSA responses in high ionic strength PB solutions, that further prove porous PEG modification can effectively suppress the Debye screening effect. Furthermore, the tunable PEG/ethanolamine ratio in this example demonstrates the good designability and universality of this modification strategy for detecting various analytes with different sizes.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article, comprising:
a field-effect transistor comprising a gate comprising a nanoscale wire at least partially coated with a polymer having an average pore size of between about 2 nm and about 10 nm.

2. The article of claim 1, wherein the polymer comprises polyethylene glycol.

3. The article of any one of claim 1 or 2, wherein the polymer comprises polypropylene glycol.

4. The article of any one of claims 1-3, wherein the average pore size is between about 2 nm and about 5 nm.

5. The article of any one of claims 1-4, wherein the average pore size is between about 5 nm and about 10 nm.

6. The article of any one of claims 1-5, wherein the average pore size is determined visually using SEM.

7. The article of any one of claims 1-6, wherein the polymer has a thickness of less than about 1 nm.

8. The article of any one of claims 1-7, further comprising a reaction entity immobilized relative to the nanoscale wire.

9. The article of claim 8, wherein the reaction entity comprises an antibody.

10. The article of any one of claim 8 or 9, wherein the reaction entity comprises a protein.

11. The article of any one of claims 8-10, wherein the reaction entity comprises an enzyme.

12. The article of any one of claims 8-11, wherein the reaction entity comprises a nucleic acid.

13. The article of any one of claims 8-12, wherein the reaction entity is covalently immobilized to the nanoscale wire.

14. The article of any one of claims 8-13, wherein the reaction entity is immobilized to the nanoscale wire via a linker.

15. The article of any one of claims 8-14, wherein the reaction entity is bound to the nanoscale wire via a carboxyl linker.

16. The article of any one of claims 1-15, wherein the nanoscale wire is a nanowire.

17. The article of any one of claims 1-15, wherein the nanoscale wire is a nanotube.

18. The article of any one of claims 1-15, wherein the nanoscale wire is a carbon nanotube.

19. The article of any one of claims 1-15, wherein the nanoscale wire is graphene.

20. The article of any one of claims 1-15, wherein the nanoscale wire comprises silicon.

21. The article of any one of claims 1-15, wherein the nanoscale wire comprises a metal oxide.

22. The article of any one of claims 1-15, wherein the nanoscale wire consists essentially of silicon.

23. The article of any one of claims 1-15, wherein the nanoscale wire comprises p-type silicon.

24. The article of any one of claims 1-23, wherein the nanoscale wire has a length of at least about 100 nm.

25. The article of any one of claims 1-24, wherein the nanoscale wire has a cross-sectional diameter of less than about 100 nm.

26. The article of any one of claims 1-25, wherein the nanoscale wire has a cross-sectional diameter of less than about 30 nm.

27. The article of any one of claims 1-26, wherein the nanoscale wire is a single crystal.

28. The article of any one of claims 1-27, wherein the nanoscale wire has an aspect ratio of at least 4:1.

29. The article of any one of claims 1-28, wherein the source and drain are defined on a substrate.

30. The article of claim 29, wherein the substrate is a silicon substrate.

31. An article, comprising:
a field-effect transistor comprising a gate comprising a nanoscale wire, the nanoscale wire at least partially coated with polyethylene glycol.

32. An article, comprising:
an ionic solution comprising an analyte; and
a field-effect transistor exposed to the ionic solution, the field effect transistor comprising a gate comprising a nanoscale wire, the nanoscale wire at least partially coated with a polymer permeable to the analyte, wherein the polymer has an average pore size of between about 2 nm and about 10 nm.

33. A method, comprising:
exposing an analyte to a nanoscale wire at least partially coated with a polymer, the polymer having an average pore size of at least the average size of the analyte and no more than 120% of the average size of the analyte, wherein the analyte has an average size of at least about 2 nm.

34. The method of claim 33, wherein the average pore size is no more than 110% of the average size of the analyte.

35. An article, comprising:
a field-effect transistor comprising a gate comprising graphene at least partially coated with a polymer, the polymer containing pores having an average diameter of between about 5 nm and about 50 nm.

36. The article of claim 35, wherein the polymer comprises polyethylene glycol.

37. The article of any one of claim 35 or 36, wherein the polymer comprises polypropylene glycol.

38. The article of any one of claims 35-37, wherein the average pore size is between about 2 nm and about 5 nm.

39. The article of any one of claims 1-38, wherein the average pore size is between about 5 nm and about 10 nm.

40. The article of any one of claims 1-39, wherein the average pore size is determined visually using SEM.

41. The article of any one of claims 1-40, wherein the polymer has a thickness of less than about 1 nm.

42. The article of any one of claims 1-41, further comprising a reaction entity immobilized relative to the nanoscale wire.

43. The article of claim 42, wherein the reaction entity comprises an antibody.

44. The article of any one of claim 42 or 43, wherein the reaction entity comprises a protein.

45. The article of any one of claims 42-44, wherein the reaction entity comprises an enzyme.

46. The article of any one of claims 42-45, wherein the reaction entity comprises a nucleic acid.

47. The article of any one of claims 42-46, wherein the reaction entity is covalently immobilized to the nanoscale wire.

48. The article of any one of claims 42-47, wherein the reaction entity is immobilized to the nanoscale wire via a linker.

49. The article of any one of claims 42-48, wherein the reaction entity is bound to the nanoscale wire via a carboxyl linker.

50. The article of any one of claims 42-49, wherein the nanoscale wire has a cross-sectional diameter of less than about 100 nm.

51. The article of any one of claims 42-50, wherein the nanoscale wire has a cross-sectional diameter of less than about 30 nm.

52. The article of any one of claims 42-51, wherein the nanoscale wire has a length of at least about 100 nm.

53. The article of any one of claims 42-52, wherein the source and drain are defined on a substrate.

54. The article of claim 53, wherein the substrate is a silicon substrate.

55. A method, comprising:
exposing a protein to a field-effect transistor comprising a gate comprising graphene at least partially coated with a polymer, the polymer containing pores having an average diameter that is between 80% and 120% of the average diameter of the protein, whereby insertion of the protein into the pores alters the gating properties of the carbon nanotube.

56. The method of claim 55, wherein the average pore size is no more than 110% of the average size of the analyte.
